(19) United States
(12) Patent Application Publication
(10) Pub. No.: US 2015/0038897 A1
(43) Pub. Date: Feb. 5, 2015

(54) Low-Profile Microneedle Patch Applicator

(71) Applicant: Zosano Pharma, Inc., Fremont, CA (US)

(72) Inventors: Peter E. Daddona, Menlo Park, CA (US); Cedric Wright, Mountain View, CA (US); Matt Vargas, San Jose, CA (US); Vivian Lee, Sunnyvale, CA (US); Jessica Heasley, Mountain View, CA (US)

(21) Appl. No.: 14/447,309

(22) Filed: Jul. 30, 2014

Related U.S. Application Data
(60) Provisional application No. 61/860,001, filed on Jul. 30, 2013.
(60) Provisional application No. 61/864,857, filed on Aug. 12, 2013.

(51) Int. Cl. A61M 37/00

(52) U.S. Cl. CPC A61M 37/0015; A61M 2037/0023; A61M 2037/0046; A61M 2037/0061
USPC 604/46

(57) ABSTRACT

A low-profile applicator, and a method of manufacturing and use thereof, for impacting microneedles against the stratum corneum of a person having a housing, a diaphragm member having the microneedles, a folding member having interlinking members that hinge-ably rotate with a force member, operatively attached thereto, between a resting position and an extended position. The folding member translates energy stored within the force member when release while retaining the force member in an energized state when in the resting position.

### CROSS-REFERENCE TO RELATED APPLICATIONS

This application claims the benefit of U.S. Provisional Application No. 61/860,001, filed Jul. 30, 2013 and U.S. Provisional Application No. 61/864,857, filed Aug. 12, 2013, the entire contents of which are incorporated herein by reference.

### TECHNICAL FIELD

The present invention relates to an apparatus and method for applying a penetrating member to the stratum corneum by impact, and more particularly, the invention relates to the use of a low-profile applicator device providing an impact to penetrate the stratum corneum with a microprotrusion array for delivery or sampling of an agent.

### BACKGROUND ART

A large number of people carry drugs and therapeutic agents packaged in an applicator for periodic or emergency use to maintain their health. For example, an insulin user at risk of diabetic hypoglycemia may carry a rescue kit that provides an emergency injection of, for example, glucagon, to facilitate the release of stored glucose back into the bloodstream. Such rescue kits traditionally employ hypodermic needles, which are bulky and subject to breakage.

Percutaneous and transdermal delivery of peptides and proteins to the human body via microneedles or micro-pins provides an alternative to hypodermic injection. Transdermal delivery generally refers to a passage of an agent across the skin layers by delivering an agent (e.g., a therapeutic agent such as a drug) through the skin to the local tissue or systemic circulatory system without substantial cutting or piercing of the skin, such as with a hypodermic needle, thereby eliminating the associated pain and reducing the risk of infection. To produce a desired therapeutic effect, an applicator of the microneedles has to apply an impact speed and energy to achieve effective penetration of the stratum corneum. Providing consistent application of the microneedles allows for the delivery of controlled dosages of the therapeutic agent into the skin for systemic and local absorption.

It is known in the art to use an applicator that comprises a flexible member for microneedle transdermal delivery in a low profile system. U.S. Pat. No. 8,267,889, for example, discloses usage of a flexible metal or plastic to generate piston velocity in a low profile applicator. Flexible metal or plastic, however, are limited in displacement due to geometry. Additionally, the flexible member is subjected to deformation, often referred to as creep, from long-term exposure to high level of stresses when under load. As such, there may be a trade-off between the effective lifespan of the apparatus and the amount of energy that it may store.

It is desirable to provide a transdermal applicator that is low-profile, safe to carry, effective over a long shelf-life, and effective over a broad range of displacement, as a applicator for therapeutic agents and drugs delivered via microneedles.

### BRIEF DESCRIPTION OF THE DRAWINGS

The foregoing features of embodiments will be more readily understood by reference to the following detailed description, taken with reference to the accompanying drawings, in which:

FIG. 1 schematically illustrates a low-profile microneedle applicator according to an embodiment of the invention;

FIG. 2 schematically illustrates a low-profile microneedle applicator according to anther embodiment of the invention;

FIG. 3 schematically illustrates an operation of the folding member and the force member according to an embodiment;

FIG. 4 schematically illustrates a low-profile microneedle applicator configured to normalize the stratum corneum for microneedles delivery according to an embodiment;

FIG. 5 schematically illustrates an applicator with a three-arm folding member in an folded state according to an embodiment of the invention;

FIG. 6 schematically illustrates the applicator of FIG. 5 in an unfolded state according to an embodiment of the invention;

FIG. 7 illustrates a method of operation according to an embodiment;

FIG. 8 illustrates a method of assembly of the applicator according to an embodiment;

FIGS. 9-10 schematically show the energized and de-energized state of a low-profile applicator configured with torsional springs as the force member according to an embodiment of the invention;

FIG. 11 schematically illustrates a detail view of the torsional spring and mounting members;

FIGS. 12-13 schematically illustrate detail views of alternate embodiments of the mounting members of FIG. 11;

FIG. 14 schematically illustrates the torsional spring with a cover according to an alternate embodiment;

FIG. 15 schematically illustrates a torsional spring with two winding sections according to a preferred embodiment;

FIGS. 16A, 16B, 17A, and 17B schematically show the energized and de-energized states of a low-profile applicator configured with compression springs as the force member according to an embodiment;

FIGS. 18-19 schematically illustrate partial views of an applicator in the energized and de-energized states according to another embodiment;

FIGS. 20-21 schematically illustrate embodiments of mounting members for the compression springs of FIGS. 16A, 16B, 17A, and 17B;

FIGS. 22-23 schematically illustrate an applicator configured with extension springs as the force member according to an embodiment;

FIGS. 24-25 schematically illustrate the applicator configured with extension springs as the force member according to an alternate embodiment;

FIGS. 26-27 schematically illustrate mounting members for attaching the extension springs of FIGS. 22-25;

FIGS. 28-29 schematically illustrate the applicator configured with extension springs as the force member according to another alternate embodiment;

FIGS. 30-31 schematically illustrate the applicator configured with a concaved leaf spring as the force member according to an embodiment;

FIG. 32 schematically illustrates a profile of a concave leaf spring threaded with the center portion of the folding member;

FIG. 33 schematically illustrates the applicator configured with a wave spring as the force member according to an embodiment;

FIG. 34 shows a detail view of a wave spring;

FIG. 35 schematically illustrates a bottom view of an assembled applicator according to a preferred embodiment of the invention;

FIG. 36 schematically illustrates a bottom view of the applicator of FIG. 35 unassembled;

FIG. 37 schematically illustrates a top view of the applicator of FIG. 35;

FIG. 38 schematically illustrates a top view of the applicator of FIG. 35 unassembled;

FIG. 39 schematically illustrates a top view of the applicator of FIG. 35 in the energized state;

FIGS. 40-41 schematically illustrate a cut-out view of the applicator of FIG. 35 in the energized and de-energized state;

FIG. 42-43 schematically illustrate docking mechanisms as alternate embodiments of the retaining member;

FIGS. 44-46 illustrate exemplary embodiments of the microneedles;

FIG. 47 illustratively shows an illustrative impact force profile of the applicator;

FIGS. 48-50 illustrate the frangible section of the diaphragm member according to various embodiments;

FIG. 51 illustratively shows an unassembled view of the frangible section in the retaining member according to an illustrative embodiment; and

FIG. 52 illustratively shows an assembled view of the retaining member of FIG. 51.

FIGS. 53-55 show a measuring fixture (FIG. 53), built from the combination of a spring fixture (FIG. 54) and torque screwdriver (FIG. 55). The measuring fixture measures torque (τ) values at corresponding angular displacements (θ) for use in calculating the released potential energy of a torsion spring.

FIG. 56 illustrates a compression spring. The spring stores no energy at its free length/neutral position (e.g., free length=1.0 m), shown all the way to the left. The block at the top of the spring is assumed to be massless and therefore not exerting any compressive force on the spring. When the spring is compressed, it stores energy relative to its displacement (PE=½kx²), where the term “x” is the spring's displacement from its free length. For example, if the spring is compressed from its free length of 1.0 m to a compressed length of 0.8 m, the displacement, x, is 0.2 m (1.0 m−0.8 m=0.2 m). By way of another example, to calculate the energy change when the spring changes from a compressed length of 0.7 m (x₁ x=0.3 m) to a compressed length of 0.8 m (x₂=0.2 m):

ΔKE=½x₂²−½kxₐ²=½k(0.2 m)²−½k(0.3 m)²=−½k(0.05 m)².

### DETAILED DESCRIPTION OF SPECIFIC EMBODIMENTS

Definitions. As used in this description and the accompanying claims, the following terms shall have the meanings indicated, unless the context otherwise requires:

The term “direction” refers to a path in a coordinate system, which includes linear and angular path.

The term “hinge-ably rotate” refers to an act of rotation at a hinge or joint.

The term “operatively attach” refers to at least two separate and distinct bodies attached to one another and operating in conjunction as a single body.

The term “traverse” refers to two non-aligned axes, or non-parallel planes and surfaces.

Embodiments of the invention disclose a novel mechanism to translate a mechanical force member having energy stored in a first direction into impact energy in a second direction thereby significantly reducing the size of an applicator of a microneedle system coated with a therapeutic agent or drug. Existing linear actuators store energy in the vertical direction and, thus, have a higher profile. The mechanism also employs separate members for energy storage and for guiding the release of the energy that provides a long displacement length in a small form factor.

The present invention provides a coating formulation containing a biologically active agent which when coated and dried upon one or more microprojections forms a coating with a stabilized coating and enhanced solubilization of the coating upon insertion into the skin. As used herein, the terms “microprojections” and “microprotrusions” are used interchangeably with microneedles. The present invention further includes a device having a plurality of stratum corneum-piercing microprojections extending therefrom. The microprojections are adapted to pierce through the stratum corneum into the underlying epidermis layer, or epidermis and dermis layers. The microprojections have a dry coating thereon which contains the biologically active agent. Upon piercing the stratum corneum layer of the skin, the agent-containing coating is dissolved by body fluid (intracellular fluids and extracellular fluids such as interstitial fluid) and released into the skin for local or systemic therapy.

The solid coating is obtained by drying a formulation on the microprojection, as described in U.S. Patent Application Publication No. 2002/0128599. The formulation is usually an aqueous formulation. In a solid coating on a microprojection array, the drug is typically present in an amount of less than about 1 mg per unit dose. With the addition of excipients, the total mass of solid coating is less than 3 mg per unit dose. The microprojection array is usually present on an adhesive backing, which is attached to a disposable polymeric retainer ring. This assembly is packaged individually in a pouch or a polymeric housing.

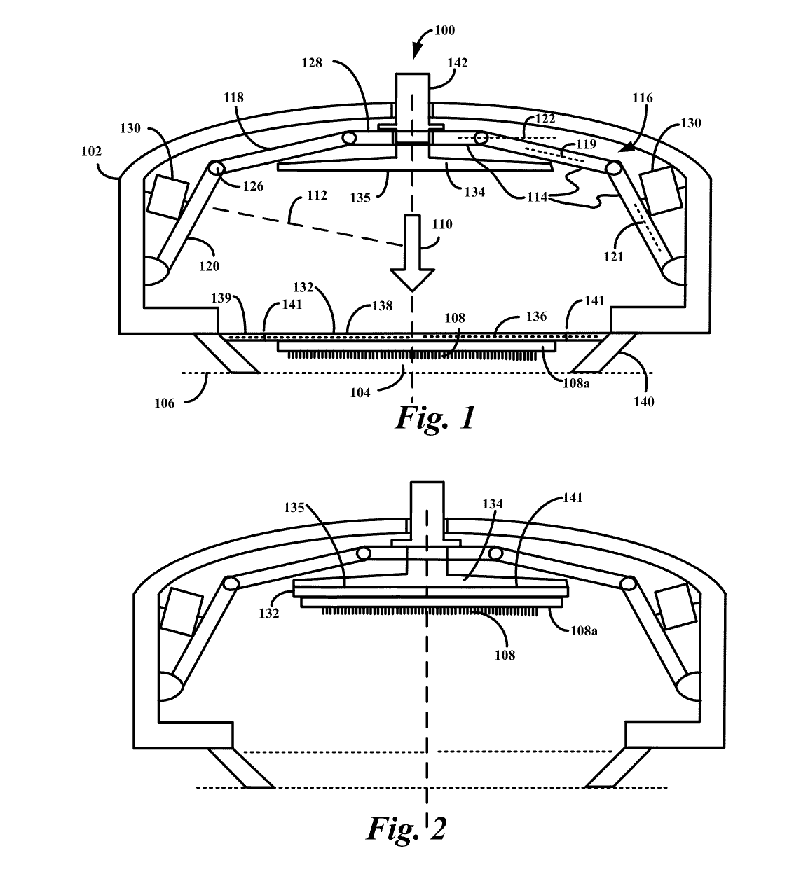

FIG. 1 schematically illustrates a low-profile microneedle applicator according to an embodiment of the invention. The applicator 100 comprises a housing 102 having an opening 104 that defines a plane 106 (referred to as an “impact plane”) for a plurality of microneedles 108 to impact and pierce the stratum corneum 109 (not shown—FIG. 4). The housing 102 may be short in a first direction 110 corresponding to the direction of the impact delivery while wide in a second direction 112 corresponding to the direction of energy storage, thereby forming a low-profile body.

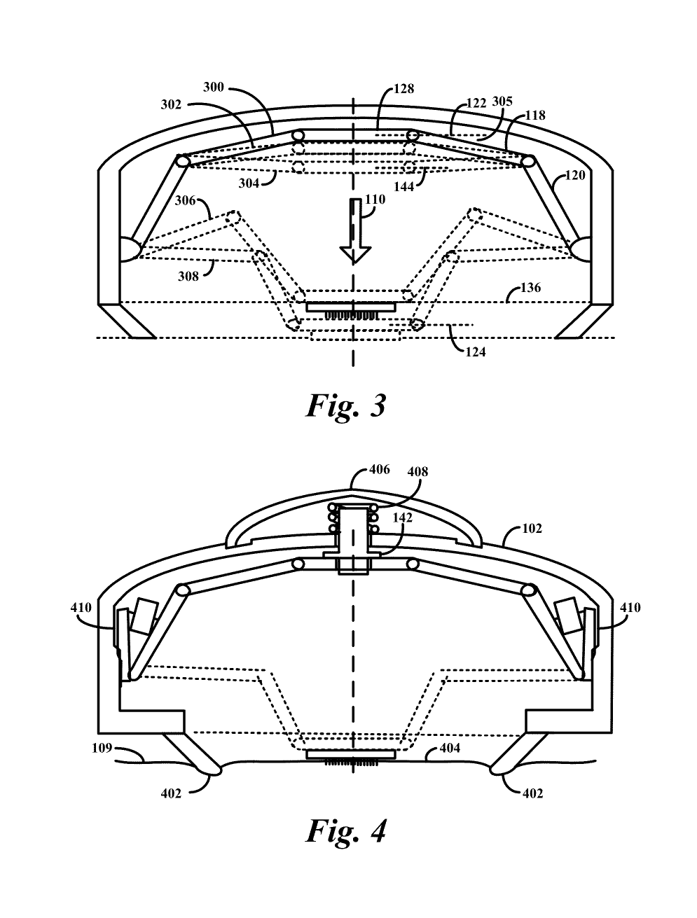

The applicator 100 includes a folding member 114 fixably disposed within the housing 102 via a plurality of arms 116. Each of the arms 116 comprises a plurality of interlinking members (e.g., 118, 120) to hinge-ably rotate between a resting position 122 and an extended position 124 (not shown—see FIG. 3). The interlinking members 118, 120, for example, may have a hinge assembly or a flexible joint connecting one another. The hinge assembly or flexible joint allows the interlinking members 118, 120 to hinge-ably rotate along a single axis 126. The folding member 114 may hinge-ably connect to the housing 102 at the interlinking members 120 and may include a central portion 128 to which the plurality of arms 116 may attach to form the folding member 114. At the resting position 122, the first section member 118 may be situated on a first plane 119, while the second section member 120 may be situated on a second plane 121 different from the first plane 119. In a preferred embodiment, the folding member 114 may include three arms. Each arm comprises a connecting link and a crank link as parts of the interlinking members.

Each respective section of the interlinking members (e.g., section member 118 and section member 120) of an arm may have the same length as corresponding sections of other arms. The symmetry of the folding member 114 allows for the symmetrical movement of the folding member 114 and the energy release of force member 130 thereby allowing for the constrained linear movement of the center portion 128 and piston 134 between the resting position 122 and the extended position 124. The folding member may be made of, for example, thermoplastics, such as polypropylene and polyethylene, among others.

The applicator 100 includes a force member 130 operatively attached to the folding member 114. The force member 130 may store energy along the second direction 112 traverse of the first direction 110 while being retained in that energized state by the folding member 114. For example, the first direction 110 may be along an axis longitudinal to the applicator 100, while the second direction 112 has an angular or radian component to that axis. In another example, the second direction 112 may be along an axis substantially perpendicular to the first direction 110. Here, the term “substantially” refers to having a variation up to 15 degrees. Of course, the second direction 112 may have other orientation with respect to the first direction 110.

The force member 130 is selected to apply a force to the folding member 114 to achieve the predetermined impact of the microneedles 108 against the stratum corneum 109. In some embodiments of the invention, the impact energy may be between 0.05 and 3 joules per cm² over a penetration period of 10 milliseconds or less. The penetration period is defined by the period of time from the initial contact with the stratum corneum 109 with the microneedles 108 to the cessation of the penetration. In certain implementations, the impact energy and velocity allows microneedles arranged in an array to penetrate a depth between 100 and 300 micrometers (microns) through the stratum corneum 109.

The force member 130 may be a single spring symmetrically disposed on the folding member 114 or a plurality of springs or bands disposed among the plurality of arms 116. In embodiments where the force member 130 is a spring, the force member 130 may be configured to be in compression or in tension in the energized state. The force member 130 may release, in the extended position, to a state where a substantial portion of the energy stored therein (i.e., at least more than half) has been expended or to a state in which some of the energy is retained with the force member 130 under slight compression or tension. Various types of springs may be employed, including, for example, torsion spring, coil springs, flat springs, planar leaf springs, disc springs, and wave springs. In other embodiments, the force member 130 may be a tension band.

Each force member stores energy through displacement from its neutral/zero energy state (e.g., displacement from free length for a linear spring). In this neutral/zero energy state, nothing is compressing, extending, or twisting the force member—it is unconstrained. The extended position of the folding member is reached when the device is at its low energy state, near (but not necessarily at) its neutral/zero energy state.

The folding member 114 and force member 130 operate in combination such that the plurality of interlinking members 118, 120 hinge-ably rotate and extend from the resting position to the extended position thereby translating a majority of the energy stored along the second direction 112 to the impact direction 110.

Examples of spring rate and torque for the force member are provided in Table 1. Of course, other rate and torque may be employed to provide impact energy between 0.05 and 3 joules per cm². For example, for torsional springs, the spring constant k may range from 0.1 to 5 in-lbs per radian.

To calculate released potential energy of a torsion spring, torque values can be measured at target rotational positions. Spring energy is equal to ½κθ², where κ is the torsion spring constant and θ is the angular displacement in radians of the spring from its free (zero energy) state. Released spring energy is the difference in stored energy between the spring's high energy state (at θ_{high}) and low energy state (at θ_{low}) in the low profile microneedle applicator (released energy=½κθ_{high}²−½κθ_{low}²).

The torsion spring constant can be calculated using torque (τ) and rotational displacement data at any two rotational positions (κ=(τ₁−τ₂)/(θ₁−θ₂)). An electromechanical torque tester may be used to measure torque (τ) values at corresponding angular displacements (θ) for use in this calculation. Alternatively, a simple measuring fixture can be made to make these measurements, as shown in FIGS. 53-55. As shown in FIG. 54, the torsion spring is placed around a cylindrical rod with the two outer ends contacting the metal piece and the inside section of the spring contacting the black piece of the fixture. As the metal piece pivots, the angular displacement of the spring changes. An indicator rigidly attached to the metal piece shows the angular displacement on the protractor. When a torque screwdriver (as shown in FIG. 55) is used to rotate the metal piece (as shown in FIG. 53), torque is indicated on the torque screwdriver while corresponding angular displacement is indicated on the protractor.

The force member may be made of a metallic alloy, for example, stainless steel, vanadium, beryllium copper, monel, Inconel, Elgiloy, NiSpan, Hastealloy, among others, as well as thermoplastic materials.

TABLE 1
Examples of spring rate and torque

| Energy | k (in-lb/rad) | Torque (in-lbs) |
|---|---|---|
| 0.26 J | 1.07 | 0.37 |
| 0.35 J | 1.44 | 0.50 |
| 0.43 J | 1.77 | 0.61 |

Equation 1 shows a simplified model of the velocity of the impact, where v is the velocity at impact (in meters per second), m is the aggregated mass impacting the stratum corneum (e.g., the center portion 128, the piston 134, the folding member 114, and a diaphragm 132) in kilograms, x_{extended} is the linear or angular displacement of the force member from the neutral/zero energy state (in meters or radians) at impact, x_{resting} is the linear or angular displacement of the force member from the neutral/zero energy state (in meters or radians) at release, and n is the number of force members (i.e., springs or bands) operatively attached to the folding member 114. The equation may be further refined by accounting for kinetic losses and the geometry of the applicator.

$$v = \sqrt{\frac{nk ( {x}_{resting}^{2} - {x}_{extended}^{2} )}{m}} \qquad ( Equation 1 )$$

When the low profile microneedle applicator is in its initial resting state, the folding member is at (or slightly above) the toggle position, the force member is at its high energy state, the displacement of the force member from the force member's neutral/zero energy state is at the maximum, and the folding member and piston are at rest (v_{resting}=0).

When the low profile microneedle applicator is in its final extended state, the folding member is at its impact position with the skin or target surface; the force member is at its low energy state; the displacement of the force member from the force member's neutral/zero energy state is at the minimum; and the folding member and piston are at maximum velocity (v_{extended}).

$$Change in Kinetic Energy = {KE}_{extended} - {KE}_{resting} = \frac{1}{2} {mv}_{extended}^{2} - \frac{1}{2} {mv}_{resting}^{2}$$

$$Change in Potential energy = {PE}_{extended} - {KPE}_{resting} = \frac{1}{2} {nkx}_{extended}^{2} - \frac{1}{2} {nkx}_{resting}^{2}$$

$$Change in Kinetic Energy + Change in Potential energy = 0$$

$$\frac{1}{2} {mv}_{extended}^{2} - \frac{1}{2} {mv}_{resting}^{2} + \frac{1}{2} {nkx}_{extended}^{2} - \frac{1}{2} {nkx}_{resting}^{2} = 0$$

$$\frac{1}{2} {mv}_{extended}^{2} - 0 + \frac{1}{2} {nkx}_{extended}^{2} - \frac{1}{2} {nkx}_{resting}^{2} = 0$$

$$\frac{1}{2} {mv}_{extended}^{2} = \frac{1}{2} {nkx}_{resting}^{2} - \frac{1}{2} {nkx}_{extended}^{2} \\ {v}_{extended}^{2} = \frac{nk}{m} ( {x}_{resting}^{2} - {x}_{extended}^{2} ) \\ {v}_{extended} = \sqrt{\frac{nk}{m} ( {x}_{resting}^{2} - {x}_{extended}^{2} )}$$

The interlinking members 118, 120 guide the release of the force member 130, which provides the energy for the movement of the interlinking members 118, 120. The extension results in the diaphragm member 132, having the microneedles 108 disposed thereon, to move to the impact plane 106 from a plane 136 substantially parallel thereto. The central portion 128 connected to the interlinking member 118, 120 may impact the diaphragm member 132 along the first direction 110 thereby propelling the plurality of microneedles 108 to impact and pierce the stratum corneum 109.

In the preferred embodiment, the applicator 100 includes a force member 130 that operatively attaches to each arm 116 of the folding member 114. In certain embodiments, multiple force members (130) may be employed for a given arm 116, in which the force members (130) are of the same or different types. In yet other embodiments, the folding member 114 may include guiding arms, which do not include a force member 130.

The diaphragm member 132 is a flexible body, which retains the microneedles 108 and conforms with the stratum corneum 109 upon contact or impact thereto. The diaphragm member 132 may be referred to as a “peelable seal.” The microneedles 108 may be arranged in an array 108a, which is retained on the diaphragm member 132. The diaphragm member 132 may form a part of a frangible section 138 and may include an attachment member 139 to be retained in the housing 102. The diaphragm member 132 may be configured to break away from the attachment member 139 at attachment points 141. The attachment member 139 may be made of the same material as the diaphragm member 132 and is defined by perforation in the structure. The frangible section 138 may include the microneedles 108 and a portion of the diaphragm member 132, which, upon impact, breaks away and is retained on the stratum corneum 109. The diaphragm member 132 is preferably mounted distal to the opening 104 to avoid inadvertent contact of the microneedles 108 thereon with the other objects or premature impact with the stratum corneum 109. In an embodiment, the diaphragm member 132 is located between 5 and 15 mm from the opening 104.

The diaphragm member 132 may be mounted, via the attachment member 139, to the housing 102 or a body fixably attached thereto. The retaining ring 140 may define the opening 104 and is shaped to cause the stratum corneum 109 to be stretched when pressed therewith. The retaining ring 140 or the housing 102 may include a seat adapted to receive the diaphragm member 132.

The folding member 114 may include a piston 134 movable with the center portion 128 within the housing 102. The piston 134 provides a surface 135 to impact the diaphragm member 132 to drive the microneedles 108 to impact the stratum corneum 109. The piston 134 may be a part of the folding member 114 or mounted thereto. The piston 134 may be circular having a width corresponding to the opening 104 with a clearance of, for example, less than 5 mm. Of course, the piston 134 may have a width substantially smaller (e.g., less than 50% of the opening 104). The surface 135 may be flat, angled, or concave depending on the surface shape of the stratum corneum 109 when the applicator 100 is disposed thereon.

In the preferred embodiment, the piston 134 is a rigid structure providing a non-compliant surface to push against the non-piercing end of the microneedles 108 thereby driving the piercing ends of the microneedles 108 into the stratum corneum 109. The surface 135 of the piston 134 is substantially planar to the impact plane 106 when the center portion 128 of the folding member 114 is in the resting position 122 and the extended position 124. Alternatively, the piston 134 may include, in part or in whole, a compliant member to conform to the surface shape of the stratum corneum 109 in contact with the applicator 100.

Alternatively, the microneedles 108 and diaphragm member 132 may be mounted on the piston 134. FIG. 2 schematically illustrates a low-profile microneedle applicator according to another embodiment of the invention. The microneedles 108 are mounted on the surface 135 of the piston 134, for example, via adhesives or magnets. The surface 135 may define the attachment points 141. Another example of the mounting is described in PCT Publication No. WO/7002521, which is incorporated by reference herein in its entirety.

Examples of the microneedles and microprotrusions are described in U.S. Pat. No. 3,814,097; U.S. Pat. No. 3,964,482; U.S. Pat. No. 5,250,023; U.S. Pat. No. 5,279,544; U.S. Pat. No. 5,879,326; U.S. Pat. No. 6,953,589; U.S. Pat. No. 7,419,481; U.S. Pat. No. 7,556,821; U.S. Pat. No. 7,658,728; U.S. Pat. No. 7,798,987; U.S. Pat. No. 7,537,795; U.S. Publ. No. 2010/0160895; Reissue 25,637; and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, WO 98/29365, and WO 06/089285; all are incorporated by reference herein in their entirety. These devices use piercing elements of various shapes and sizes to pierce the stratum corneum. The microneedles are generally referred therein as penetrating elements and generally extend perpendicularly from a thin, flat member, such as a pad or sheet. The microneedles may be arranged in an array. Some of these microneedles have dimensions (i.e., a length and width) of about 25-400 μm and a thickness of only about 5-50 μm. Other microneedles are hollow needles having diameters of about 10 μm or less and lengths of about 50-100 μm.

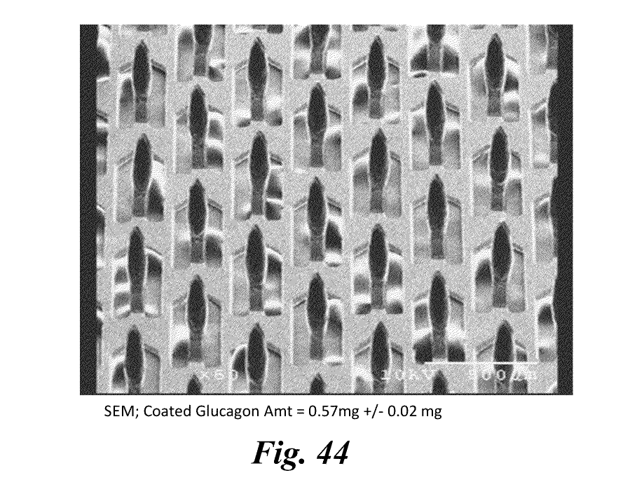

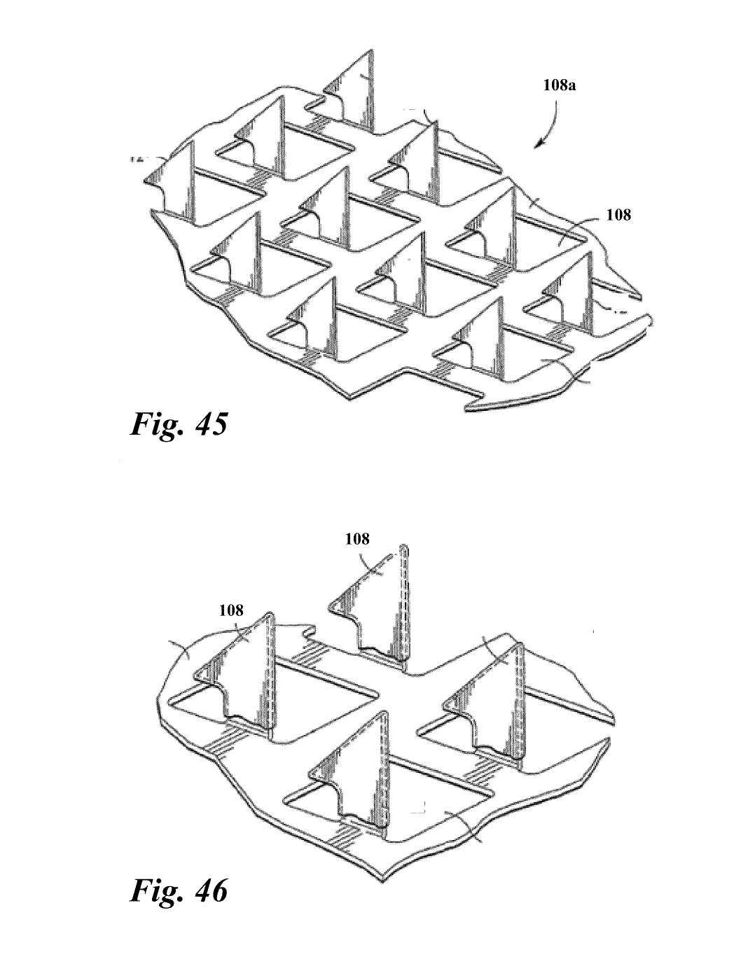

Examples of the microneedles are shown in FIGS. 44-46. In FIG. 45, the microneedles have a projection length less than 400 microns, more preferably in the range of approximately 190-400 microns. The term “length” refers to the overall length of the microneedles that may pierce into the stratum corneum.

The microneedles may have a coating or reservoir of a therapeutic agent or drug, referred to as a pharmaceutical agent. Examples of such biologically active and/or therapeutic agents include drugs, polypeptides, proteins, nucleic acids, desensitizing agents, vaccines and allergens, all of which may be natural or synthetic, derived from human or animal or other organism, and includes proteins, cytokines, cytokine receptors, enzymes, co-factors for enzymes or DNA binding proteins, polysaccharides, oligosaccharides, lipoproteins, weakened or killed viruses such as cytomegalovirus, hepatitis B virus, hepatitis C virus, human papillomavirus, rubella virus, and varicella zoster, weakened or killed bacteria such as bordetella pertussis, clostridium tetani, corynebacterium diphtheriae, group A streptococcus, legionella pneumophila, neisseria meningitides, pseudomonas aeruginosa, streptococcus pneumoniae, treponema pallidum, and vibrio cholerae and mixtures thereof.

Additional examples of such agents include, without limitation, polypeptide and protein drugs such as leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH(1-24), calcitonin, parathyroid hormone (PTH), Dihydroergotamine (DHE), vasopressin, deamino [Val4, D-Arg8] arginine vasopressin, interferon alpha, interferon beta, interferon gamma, erythropoietin (EPO), granulocyte macrophage colony stimulating factor (GM-CSF), interleukin-10 (IL-10), glucagon, and glucagon like peptide-1 (GLP-1 and analogs); analgesic drugs such as fentanyl, sufentanil, and remifentanyl; antigens used in vaccines such as influenza vaccines, Lyme disease vaccine, rabies vaccine, measles vaccine, mumps vaccine, chicken pox vaccine, small pox vaccine and diptheria vaccine; and desensitizing agents such as cat, dust mite, dog, and pollen allergens; PTH based agents including hPTH (1-34), hPTH salts and analogs, teriparatide and related peptides; hPTH salts including acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate, granulocyte colony stimulating factor (G-CSF), glucagon, growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl]carbonyl]-L-histidyl-L-prolinamide), liprecin, pituitary hormones (e.g., HGH, HMG, desmopressin acetate, etc), follicle luteoids, αANF, growth factors such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, epoprostenol (platelet aggregation inhibitor), HCG, hirulog, hyaluronidase, interferon, interleukins, oxytocin, streptokinase, tissue plasminogen activator, urokinase, ANP, ANP clearance inhibitors, angiotensin II antagonists, antidiuretic hormone agonists, bradykinin antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives, such as formivirsen, alendronic acid, clodronic acid, etidronic acid, ibandronic acid, incadronic acid, pamidronic acid, risedronic acid, tiludronic acid, zoledronic acid, argatroban; triptan compounds (sumatriptan, almotriptan, eletriptan, frovatriptan, naratriptan, rizatriptan, Zolmitriptan); and mixtures thereof.

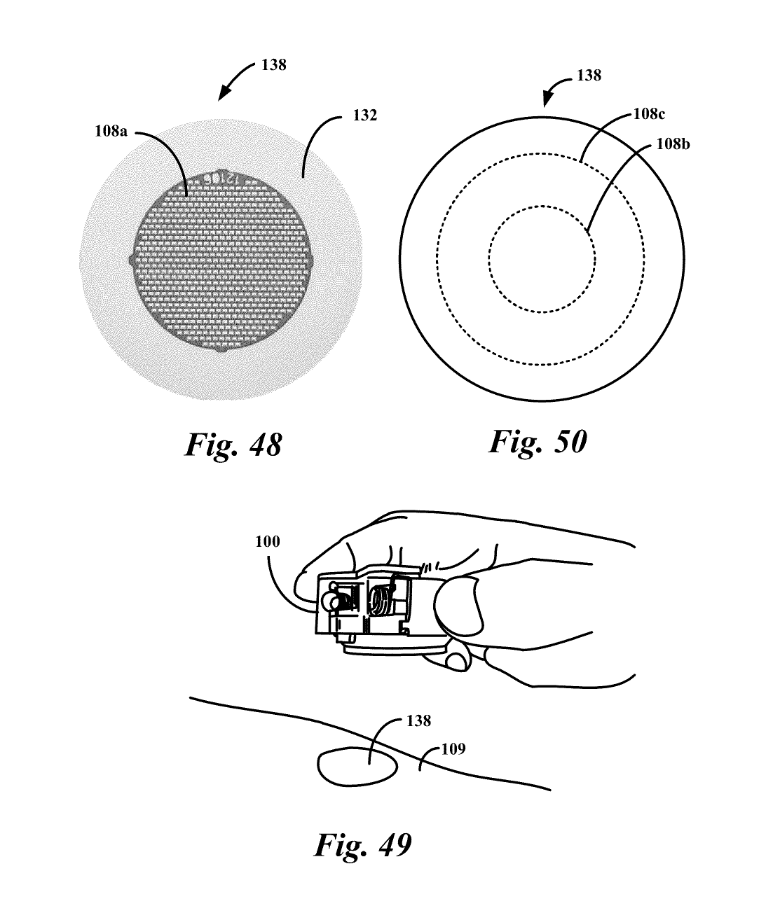

In a preferred embodiment, the frangible section 138 comprises the diaphragm member 132 to retain a microneedle array portion 108a, which is propelled to impact and pierce the stratum corneum 109 upon impact by the applicator 100. The microneedle array portion 108a may be located in the center of the frangible section 138 surrounded by an adhesive portion of the diaphragm member 132. FIGS. 48 and 49 illustrate an example of the frangible section 138. The flexible membrane portion may have adhesives to retain the frangible section 138 on the stratum corneum 109 and may be made of rubber or synthetic material, such as acrylic-based pressure sensitive adhesives (PSA). The microneedle array portion 108a includes the microneedles 108 arranged in an array as shown, for example, in FIGS. 44-46 and may be preferably made of medical-grade material, such as titanium, more preferably grade-2 titanium.

The size of microneedle array portion 108a may vary according to the intended dosage of the pharmaceutical agent to be delivered. To that end, the frangible section 138 may have the area of the microneedle array portion 108a coated with a uniform coating of the pharmaceutical agent. FIG. 50 illustrates the size of the microneedle array portion 108b, 108c with an area of about 2 cm² and 10 cm² corresponding to a diameter of about 1.6 cm and 3.6 cm. Preferably, the size of the microneedle array portion 108a is between 3 cm² and 6 cm². The 3-cm² array 108a may be retained, for example, on a 5-cm² diaphragm member 132 in a retaining member 140 having a 2 to 7 cm opening. The 6-cm² may be retained, for example, on a 10-cm² diaphragm member 132 in a retaining member 140 having a 4 to 7 cm opening. Of course, other dimensions may be employed.

The diaphragm member 132 and the microneedle array portion 108a of the frangible section 138 may have various shapes, preferably round or squares, in which they are the same or different. In certain embodiments, the frangible section 138 may have complex shapes, such as a star, an animal, a plant, and other ornate shapes.

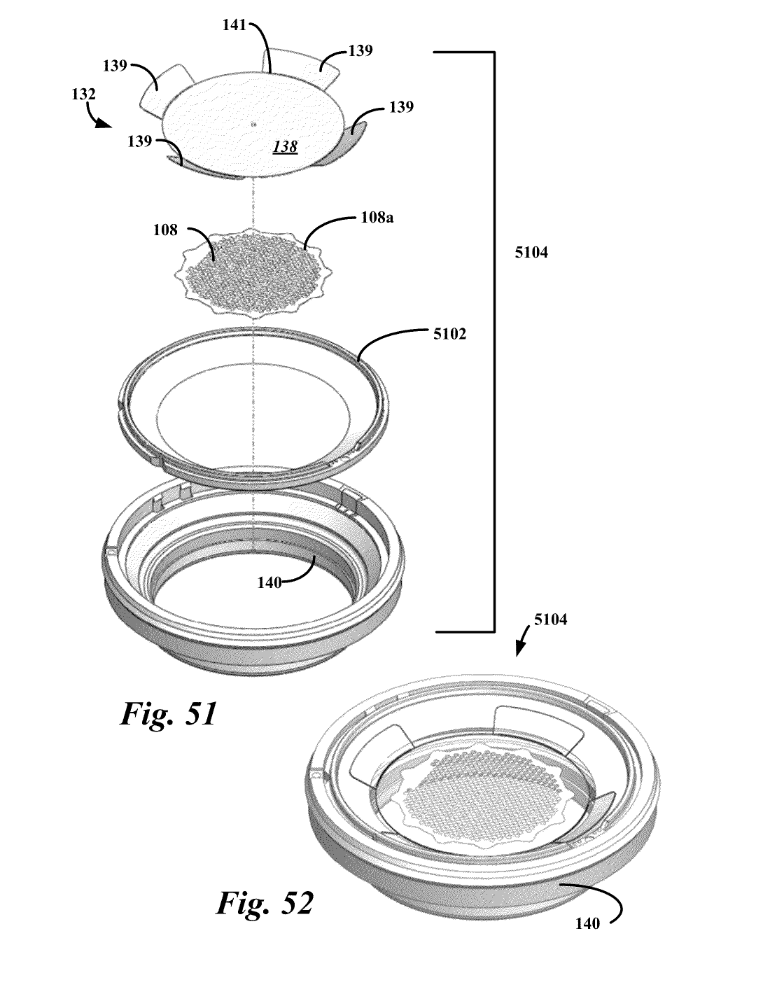

FIG. 51 illustratively shows an unassembled view of the frangible section 138 in the retaining member 140 according to an illustrative embodiment. The frangible section 138 with the microneedle array portion 108a is attached via attachment members 139 to an inner ring member 5102 to form a patch assembly 5104. The frangible section 138 is configured to break away from the attachment members 139 along the attachment points 141. The assembly 5104 may be manufactured and packaged according to the desired dosage of the pharmaceutical agent to be assembled to the applicator 100. The assembly 5104 is configured to be attached to the retaining member 140, for example, by snap fit. Of course, other attaching means may be employed, including, for example, via adhesives, sonic-welding, solvent-bonding, and mechanical linkages, such as screws, and bolts.

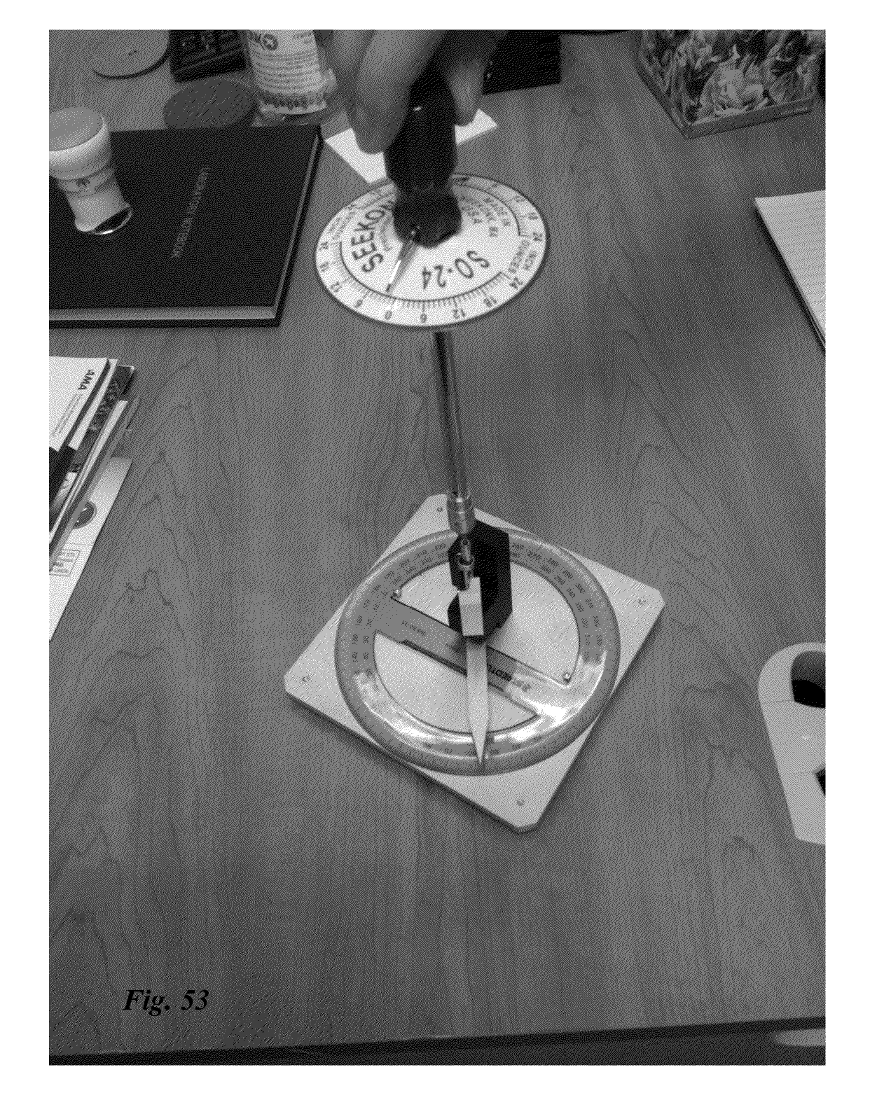

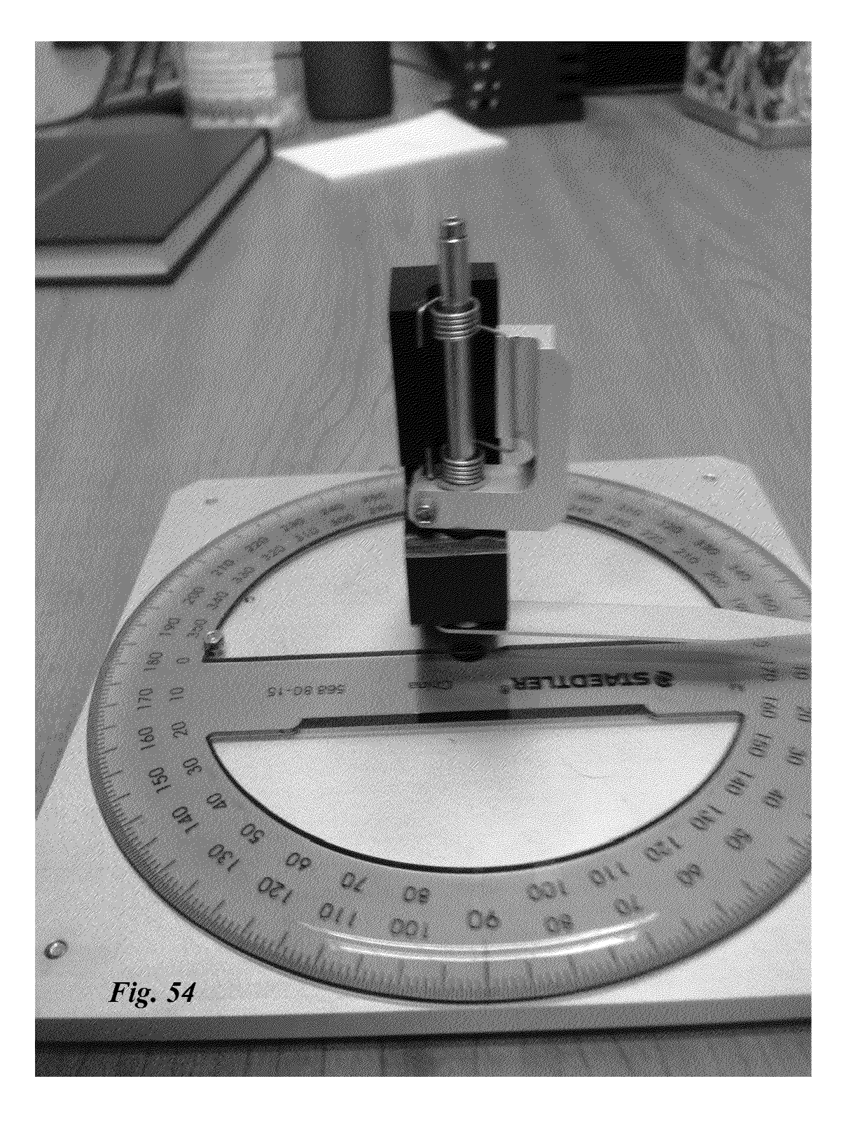

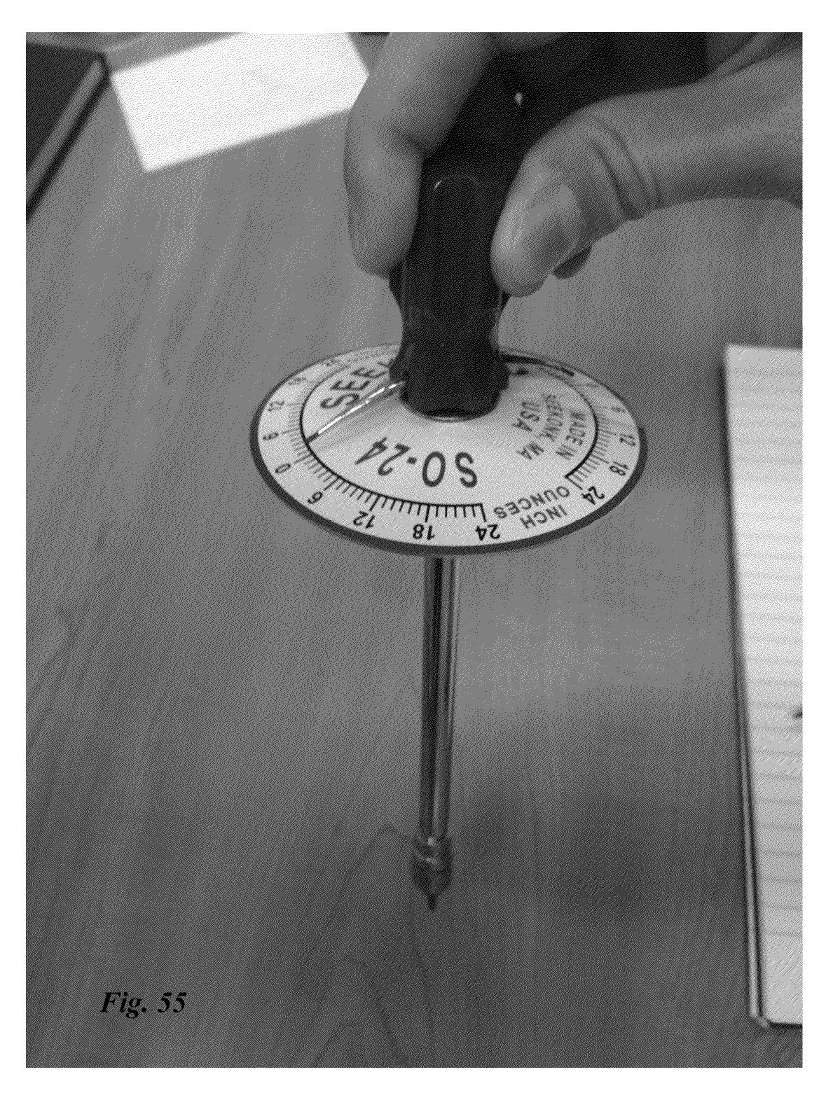

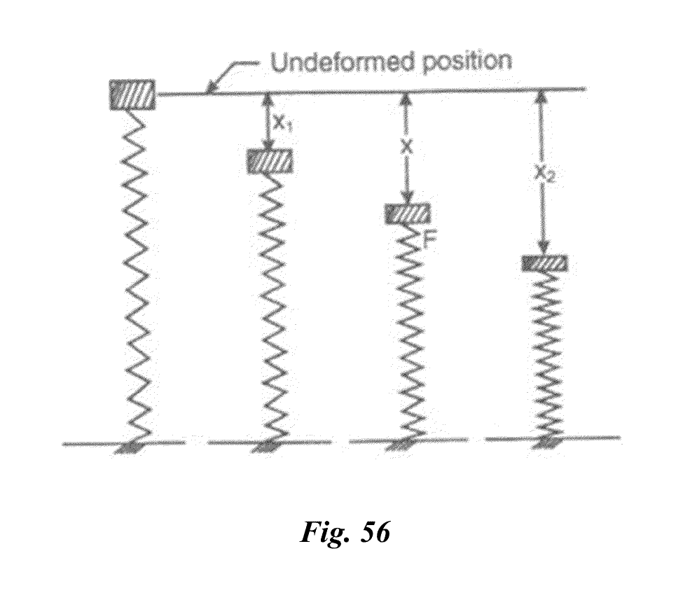

FIG. 52 illustratively shows an assembled view of the assembly 5104 of FIG. 51. The retaining member 140 may include a slot or hook to retain the inner ring member 5102. The retaining member 140 may include a hook to connect to the housing 102. Alternatively, the inner ring member 5102 may be press-fit to the bottom of the housing 102 or attached via any of the above described attachment means.

Referring back to FIG. 1, the applicator 100 may include a release member 142 to activate the delivery of the microneedles 108 against the stratum corneum 109. In an embodiment, the release member 142 provides a mechanism, by acting as a piston, to displace the folding member 114 from the resting position 122 past a toggle position 144 (not shown—see FIG. 3) in which the force member 130 is allowed to hinge-ably rotate the folding member 114.

In certain embodiments, the release member 142 may include a locking mechanism to retain the center portion 128 of the folding member 114 in the resting position 122. For example, the locking mechanism may include a cover disposed over the release member 142 to prevent the unintentional displacement or release of the folding member 114 and/or creep. The locking mechanism may be a part of the release member 142 and may include, for example, a slot and key element. In such embodiment, the release member 142 may be configured such that a user of the applicator would rotate the release member 142 to align the key element with a slot, thereby allowing the release member 142 to move from its locked position.

FIG. 3 schematically illustrates an operation of the folding member 114 and the force member 130 according to an embodiment and is described in conjunction with FIG. 1. At a resting state (shown as state 300), the folding member 114 retains the force member 130 in an energized state. Either the folding member 114 or the release member 142 may be disposed against the housing 102. This state may also be referred to as the cocked state.

Upon release of the folding member 114 by the release member 142 (shown as state 302), for example, by being activated by a person pressing thereon, the release member 142 acts as a piston to displace the center portion 128 from the resting position 122. The initial displacement of the center portion 128 may not trigger the release or activation of the applicator 100. To that end, when the center portion 128 passes the toggle position 144 (shown as horizontal state 304), the second section member 120 is then allowed to rotate along with the first section member 118 along the first direction 110. The force member 130 begins to extend, or unwind, releasing energy stored therein causing the first and second section members 118, 120 of the folding member 114 to hinge-ably rotate and accelerate the center portion 128 along the first direction 110.

In an embodiment, for example, the toggle position 144 is defined by a change in the sign (e.g., positive to negative or vice versa) of an angle 305 formed between the center portion 128 and the first member section 118.

As the center portion 128 accelerates toward the extended position 124, the piston 134 may impact the diaphragm member 132 with the microneedles 108 (shown as state 306). The diaphragm 132 breaks away as a part of the frangible section 138 to impact the stratum corneum 109. The impact by the folding member 114 may break the diaphragm member 132 from the attachment member 139 at attachment points 141.

Upon contact with the stratum corneum 109 (shown as state 308), the center portion 128 of the folding member 114 begins to decelerate while driving the microneedles 108 into the stratum corneum 109. The center portion 128 of the folding member 114 comes to rest at the extended position 124. The extended position 124 generally refers to a resting position of the center portion 128 of the folding member 114 and varies according to whether the applicator 100 is resting against the stratum corneum 109 or fully extended against no other surfaces. To effectively deliver the microneedles in the stratum corneum 109, the frangible member 138 impacts with energy between 0.05 and 3 joules per cm² within less than ten milliseconds.

Alternatively, the frangible section 138 with the microneedles 108 may break after impact with, and is retained on, the stratum corneum 109. The frangible section 138 may include a coating of adhesive to help retain the frangible section 138 on the stratum corneum 109.

In another aspect of the embodiment of the invention, the applicator 100 is adapted with features to normalize the stratum corneum for the microneedles delivery. FIG. 4 schematically illustrates the applicator configured to normalize the stratum corneum for microneedles delivery according to an embodiment. The retaining ring 140 has a curved wall 402 for contacting and stretching, when pressed against, the stratum corneum 109. In doing so, the stratum corneum 109 may dome past the opening 104 into the housing 102. The contact area may be between two and five centimeters in diameter. In the preferred embodiment, a contact force preferably less than fifteen pounds may be applied to the applicator 100. More preferably, the contact force may be between two and fifteen pounds, and even more preferably the contact force may be between five and ten pounds and, in most instances, eliminate the recoil of the applicator 100.

In another aspect of the embodiment of the invention, the applicator 100 includes a self-acting feature that triggers the activation of the microneedles delivery when a sufficient contact force is applied to the applicator 100. In an embodiment, the applicator 100 includes a flexible cover 406 mounted to the housing 102. The cover 406 may be configured to elastically deform to trigger the activation of the delivery when a force is applied sufficient to both (i) move the release member 142 past the toggle point 144 and (ii) normalize the stratum corneum 109 for the microneedles delivery. The applicator 100 may include a spring member 408 positioned between the cover 406 and the release member 142 to vary this triggering/normalizing force. The applicator 100 may have an exterior surface shaped to allow for the ergonomic application of the contact force.

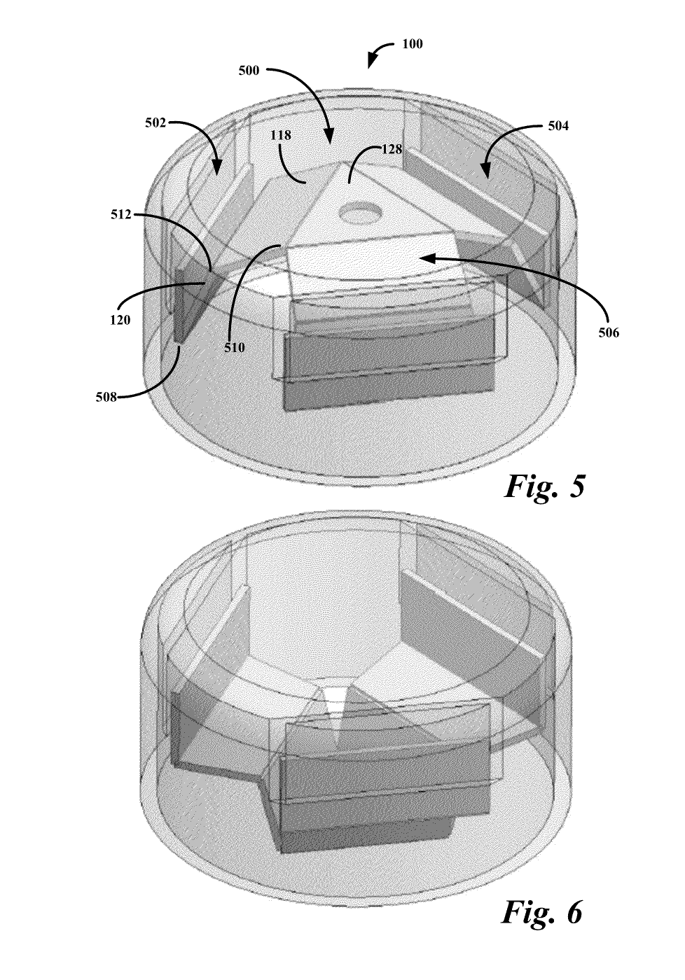

FIG. 5 schematically illustrates an applicator with a three-arm folding member 500 in a folded state according to an embodiment of the invention. In the folded state, the folding member 500 retains the force member 130 in the energized state. The housing 102 has a generally annular opening to house the folding member 114 with three arms 502, 504, 506. The arms 502, 504, 506 are symmetrically shaped and operate in conjunction with one another to hinge-ably rotate thereby propelling the center portion 128 from the resting position 122 and the extended position 124. Each arm (502, 504, 506) forms a vertical crank-slider assembly in which (i) the first interlinking member 118 (referred to as a “connecting link”) is configured to pivot or rotate at a second flexible joint or hinge assembly 510 proximal to the center portion 128 and (ii) the second interlinking member 120 (referred to as a “crank link”) is configured to pivot or rotate at a first flexible joint or hinge assembly 508 proximal to the housing 102. The first interlinking member 118 further pivots or rotates at a third flexible joint or hinge assembly 512 connected with the second interlinking member 120. Each arm (502, 504, 506) may be referred to as a four bar linkage. In the resting position, the first interlinking member 118 is generally horizontal while the second interlinking member 120 is generally vertical. The term “generally” refers to being within 1-20 degrees thereof.

FIG. 6 schematically illustrates the applicator 100 with the three-arm folding member 500 of FIG. 5 in a de-energized state according to an embodiment of the invention. The center portion 128 has toggled from the resting 122 position to the extended position 124. In the extended position, the first interlinking member 118 is generally vertical, while the second interlinking member 120 is generally horizontal.

In translating from the resting position to the extended position, the second interlinking member 120 rotates between 70 and 95 degrees at flexible joint or hinge assembly 508. During the rotation, the angle between the first interlinking member 118 and the second interlinking member 120 may initially decrease and then expand. The length of the first interlinking member 118 may be defined by the location of the flexible joint or hinge assembly 510 and the displacement of the center portion 128.

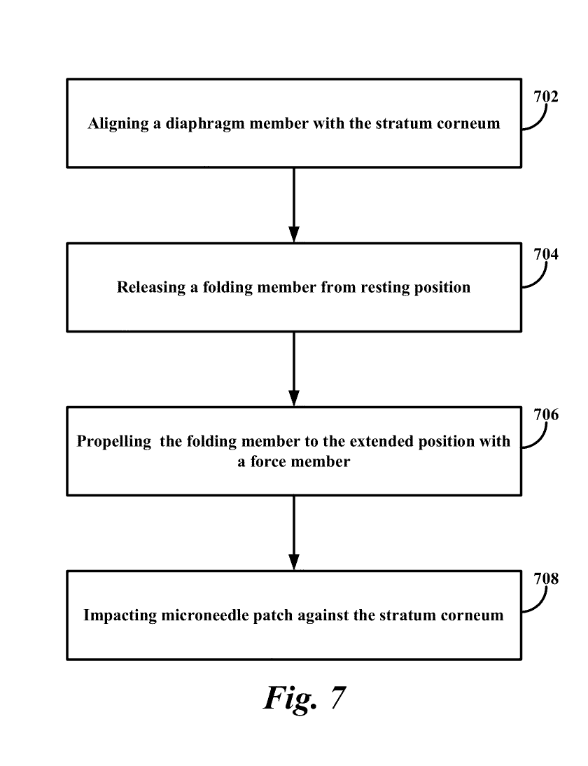

FIG. 7 illustrates a method of operation according to an embodiment. In general, the method begins by a person positioning the opening 104 of the applicator 100 against the stratum corneum thereby aligning the diaphragm member 132 substantially parallel (i.e., less than 5 degrees) with the stratum corneum 109 (step 702). The diaphragm member 132 may be attached to the folding member 114 at the piston 134 or a recess in the retaining member 140 attached to the housing 102. The person may apply a contact force between two and fifteen pounds to normalize (e.g., stretching) the stratum corneum 109 to form a dome-like bulge that extends past the opening 104 of the housing 102.

To begin the delivery of the microneedles 108 to the stratum corneum 109, the person presses on the release member 142. The release member 142 releases the folding member 114 from the resting position 122 to rotate the first interlinking member 118 (step 704). The first interlinking member 118 rotates with respect to the second interlinking member 120, which retains the force member 130. As the first interlinking member 118 rotates, the center portion 128 moves in the vertical direction passing the toggle position 144 at which the second interlinking member 120 is allowed to rotate allowing the force member 130 to extend or unwind. Of course, other mechanisms (manual or automated) may be employed to activate the release member 142. For example, the locking mechanism may retain the release member 142 in a locked state until the person intends for the microneedles delivery. The release member 142 may, for example, include a key and pin assembly, which requires the person to rotate and align the release member 142 to an unlock position to allow for the displacement of the release member 142 and, thus, the release of the center portion 128.

As the center portion 128 moves past the toggle position 144, the force member 130 extends or unwinds to propel the rotation of the second interlinking member 120 (step 706). The second interlinking member 120 is hinge-ably linked to the first interlinking member 118 to cause the movement thereof. The first interlinking member 118 is hinge-ably linked to the center portion 128, which is constrained by the other arms of the folding member 114 to move towards only the first direction 110. As the center portion 128 passes the plane 136 with the diaphragm member 132, the piston 134 impacts the frangible section 138 of the diaphragm member 132 with the momentum of the folding member 114 and the piston 134, as well as the energy of the force member 130.

As the center portion 128 of the folding member 114 approaches the extended position 124, the microneedles 108 impacts the surface of the stratum corneum 109 with an impact energy between 0.05 and 3 joules per cm² (step 708) with a penetration time less than 10 milliseconds. At the prescribed impact energy, the microneedles 108 are inserted into the stratum corneum 109 at a depth between 100 and 300 micrometers allowing therapeutic agents or drug coated on the microneedles 108 to dissolve into the interstitial fluid of the skin.

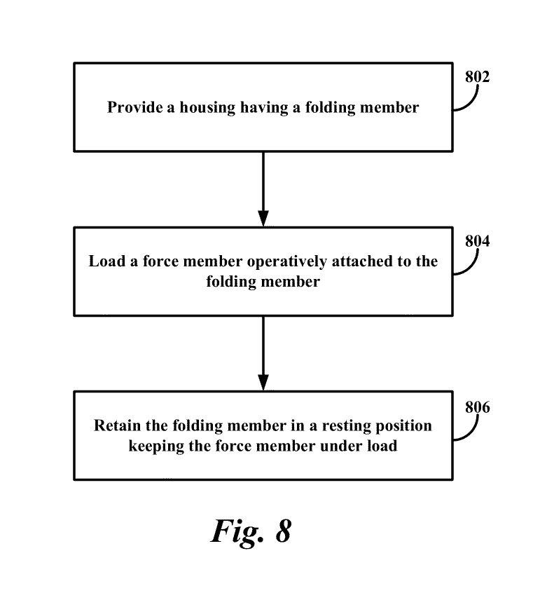

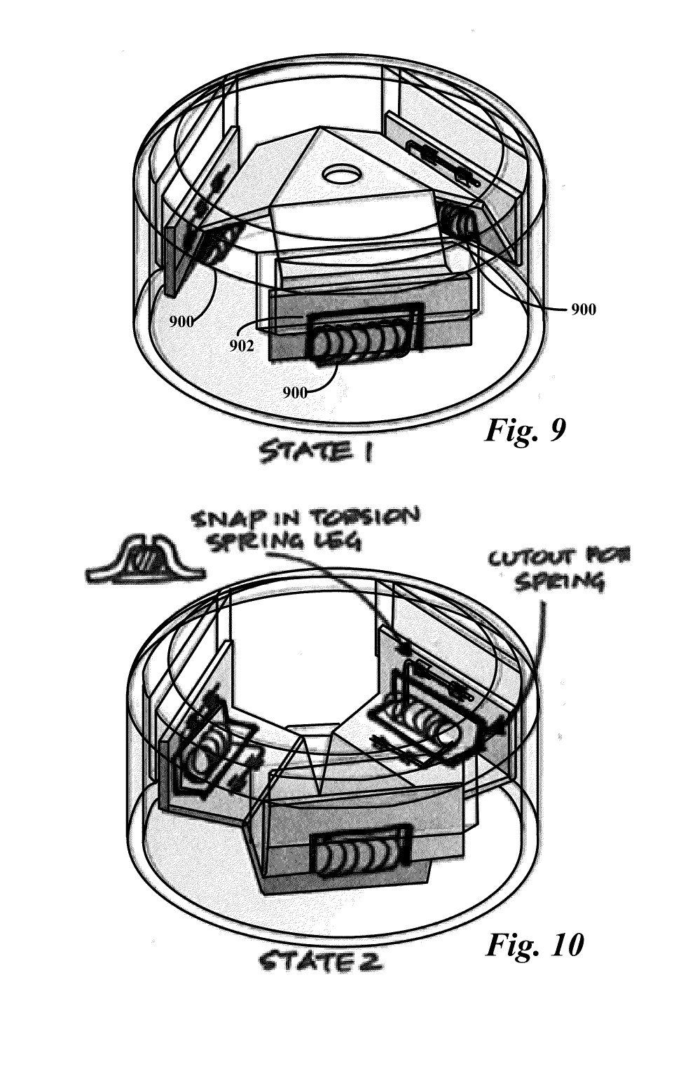

FIG. 8 illustrates a method of assembly according to an embodiment. The method begins by providing a housing 102 with the folding member 114 and the force member 130 operatively attached thereto (step 802). Examples of various folding members 114 and force member 130 and their assembly within the housing 102 are illustratively shown in FIGS. 9-33.

FIGS. 9-10 schematically show the energized and de-energized states of a low-profile applicator 100 configured with torsional springs 900 as the force member 130 according to an embodiment. The torsional spring 900 is a winding structure that elastically twists or rotates along an angular direction around the axis within its winding to store mechanical energy. Three torsional springs 900 corresponding to the number of arms 116 of the folding member 114 may be employed.

The first and second interlocking members 118, 120 of the folding member 114 may include a cut-out portion 902 for mounting the torsional spring 900. A first spring leg 904 of the torsional spring 900 fixably attaches to the first interlinking member 118, and a second spring leg 906 fixably attaches to the second interlinking member 120. The spring 900 may twist with the rotation of the first and second interlinking members 118, 120 up to 90 degrees.

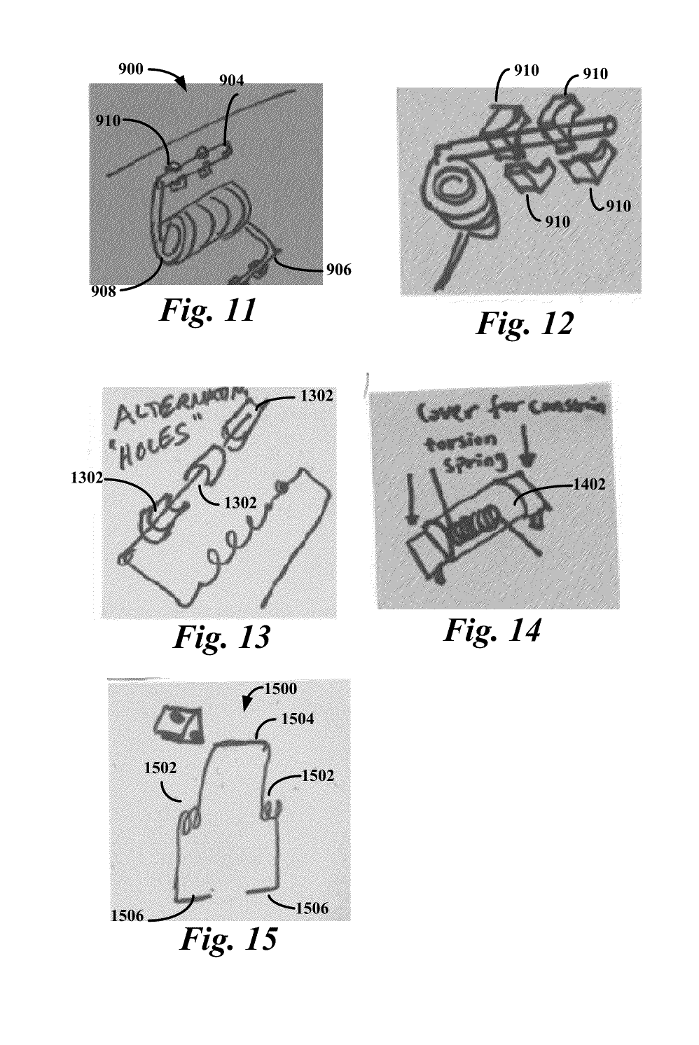

FIG. 11 schematically illustrates a detail view of the torsional spring 900 and mounting members 910. The spring 900 includes a winding section 908 with the first and second spring legs 904, 906 extending from each side. The folding member 114 may include mounting members 910 to retain the first and second 904, 906 of the torsional spring 900. The mounting members 910 may form hooks or holes to retain the spring leg between energized and de-energized state.

FIGS. 12 and 13 schematically illustrate detail views of alternate embodiments of mounting members 910 of FIG. 11. In FIG. 12, the mounting members 910 extend from the folding member 114 with a gap therebetween to form a snap fit for the first and second spring legs 906, 908 of the torsional spring 900 while allowing the spring legs to rotate. In FIG. 13, the folding member 114 is formed with alternating holes 1302. In certain embodiments, the mounting members 910 may be located on the first and second interlinking members 118, 120 to retain and operatively attach each of the torsional springs 900 to the folding member 114. In another embodiment, the mounting members 910 may be located on the housing 102 and one of the interlinking members (118, 120).

FIG. 14 schematically illustrates the torsional spring 900 with a cover 1402. The cover 1402 includes a plurality of attachment members 1404 to fixably attach to the folding member 114.

FIG. 15 schematically illustrates a torsional spring 1500 to mount to the perimeter of the folding member 114. The torsional spring 1500 have two winding sections 1502 formed from a single wire. The wire forms a center portion 1504 and an end portion 1506. The winding sections 1502 may attach to a groove formed at the hinge assembly or flexible joint allowing for the rotation to coincide with the first and second interlinking members 118, 120. The center portion 1504 may, for example, be disposed against the second interlinking member 120 or the housing 102, and the end portion 1506 is disposed against the first interlinking member 118, or vice versa.

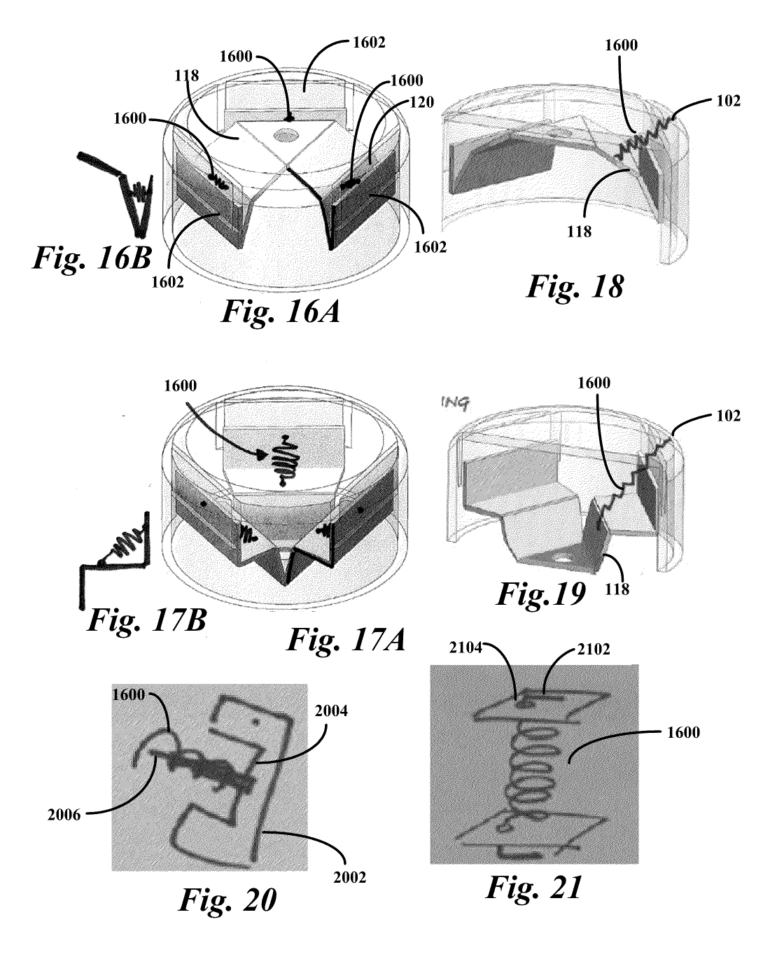

FIGS. 16A, 16B, 17A, and 17B schematically show the energized and de-energized states of a low-profile applicator 100 configured with compression springs 1600 as the force member according to an embodiment. A compression spring contracts in length along its longitudinal axis to store energy. In FIG. 16A, the compression springs 1600 are attached between portions of the folding member 114, for example, at the second interlinking members 120 and a third interlinking member 1602. FIG. 16B shows a profile view of the attachment. Various types of compression springs may be employed, including conical, barrel, constant pitch, hourglass, and variable pitch springs. FIG. 17A schematically shows the applicator of FIG. 16A in the de-energized state. FIG. 17B shows the corresponding profile of FIG. 17A.

FIGS. 18 and 19 schematically illustrate partial views of an applicator in energized and de-energized states according to another embodiment. The compression spring 1600 is attached between the first interlinking member 118 of the folding member 114 and the housing 102.

FIGS. 20 and 21 schematically illustrate mounting members of the folding member 114 or housing 102 for the compression springs of FIGS. 16A, 16B, 17A, and 17B. In FIG. 20, a mounting member is configured as a rod and cup assembly 2002 and includes a cup portion 2004 to retain the center portion of the spring and a rod portion 2006 to retain the ends of the springs. In FIG. 21, the mounting location includes a hole and lock assembly. The end portion 2102 of the compression spring 1600 is bent through a hole 2104 to retain the compression spring 1600 in place when under compression.

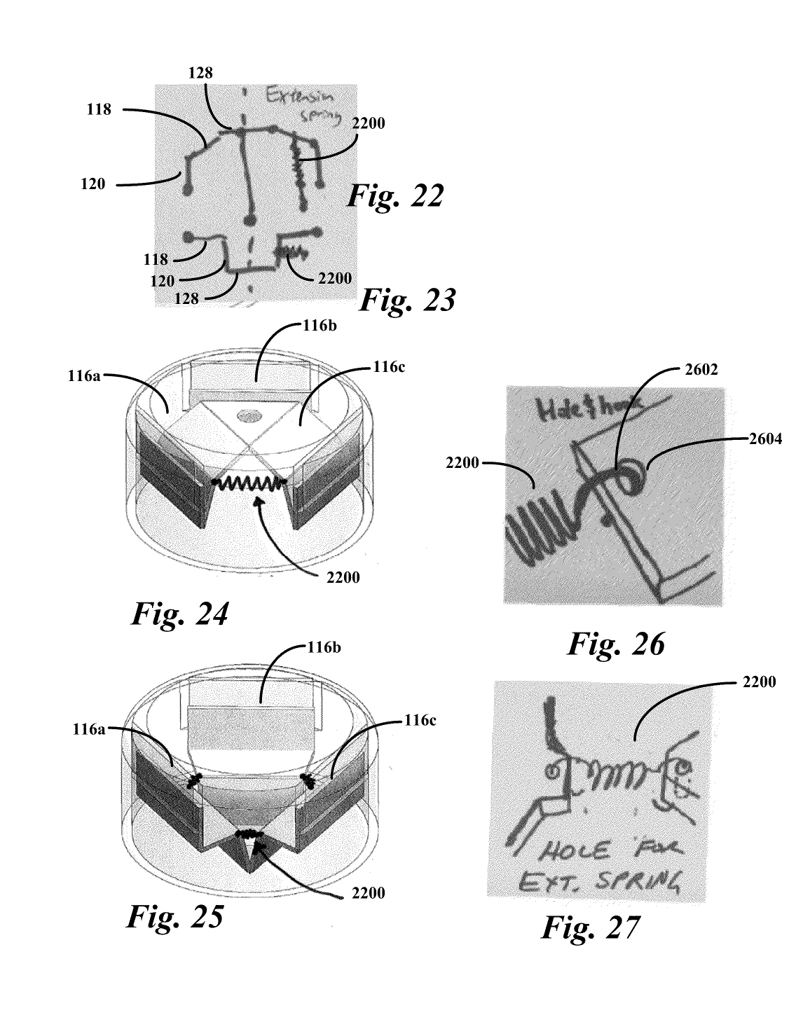

FIGS. 22 and 23 schematically illustrate an applicator configured with extension springs 2200 as the force member according to an embodiment. Rather than contracting, extension springs expands in length along its longitudinal axis to store mechanical energy. In FIG. 22, the extension spring 2200 is attached to the first interlinking member 118 and the housing 102. In the energized state, the springs 2200 are oriented in a generally vertical orientation traverse of the first direction 110. The release member 142 secures the folding member 114 to the housing 102 until release. FIG. 23 shows the force member 130 and folding member 114 in the de-energized state in which the springs 2200 are oriented in a horizontal orientation.

FIGS. 24-25 schematically illustrate the applicator configured with extension springs 2200 according to an alternate embodiment. The extension springs 2200 operatively attach between different arms 116a-c of the folding member 114. FIG. 24 shows the extension springs 2200 in an extended energized state, and FIG. 25 shows the springs 2200 in a contracted de-energized state.

FIGS. 26 and 27 schematically illustrate mounting members for attaching the extension springs 2200 of FIGS. 22-25. In FIGS. 26 and 27, the extension spring 2200 includes a hook 2602 configured to secure with a hole 2604 located at the mounting location either in the folding member 114 or the housing 102.

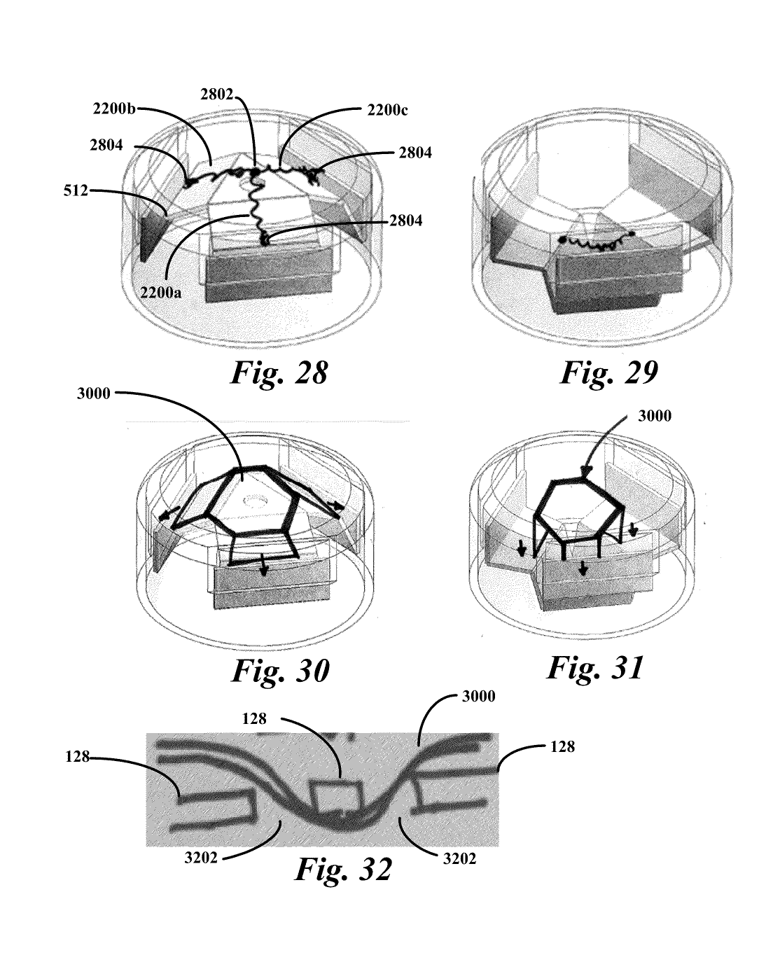

FIGS. 28-29 schematically illustrate the applicator configured with extension springs 2200 as the force member according an alternate embodiment. The extension springs (2200a, 2200b, 2200c) are linked to one another at a common leg 2802 to each other to the release member 142. Each of the extension springs 2200 includes a spring leg 2804, which attaches to the folding member 114 at either the first interlinking member 118 or the hinge or joint 512.

FIGS. 30-31 schematically illustrate the applicator configured with a concaved leaf spring 3000 according to an embodiment. The concaved leaf spring 3000 is a type of flat spring that forms a concave structure that elastically deforms to store energy. FIG. 30 shows the concaved leaf spring 3000 in an energized state, and FIG. 31 shows the spring 3000 in a de-energized state.

FIG. 32 schematically illustrates a profile of a concave leaf spring 3000 threaded with the center portion 128 of the folding member 114. The center portion 128 forms a radial hole 3202 for the spring 3000 to thread therethrough.

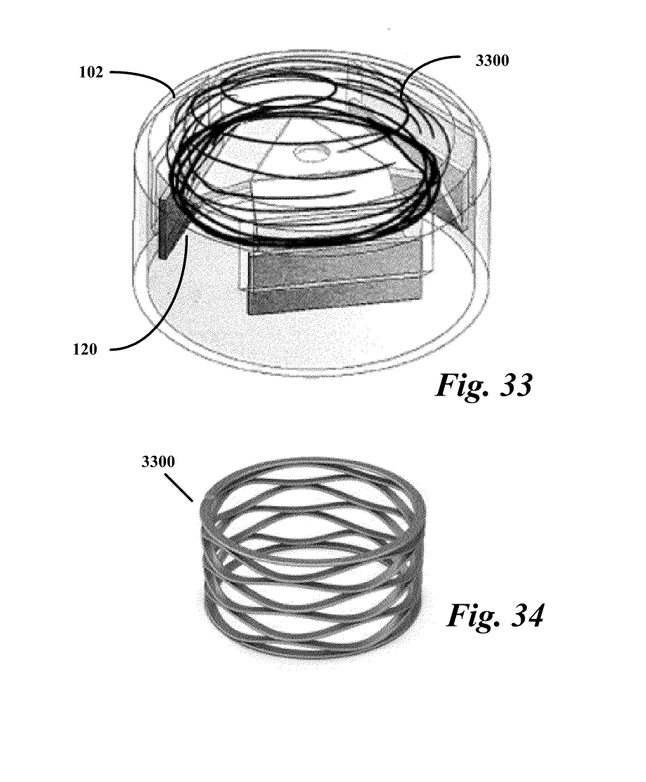

FIG. 33 schematically illustrates the applicator configured with a wave spring 3300 according to an embodiment. The wave spring 3300 forms a plurality of compressible sections staggered along the longitudinal axis of the spring that elastically compress to store energy. The wave spring 3300 may be attached between the second interlinking member 120 and the housing 102. FIG. 34 shows an example of a wave spring 3300. Of course, other types of springs or band may be employed, such as disc springs, among others.

Referring back to FIGS. 4 and 8, in another aspect of the embodiment of the invention, the housing 102 may include a recess 410 to retain and align the folding member 114 within the housing 102 during assembly (step 802). The folding member 114 may include the third interlinking member 1602 to align with the recess 410.

During assembly, the force member 130 may be operatively attached to the folding member 114 with the force member 130 in the de-energized state. The folding member 114 may be folded and placed within the housing 102 such that the third interlinking member 1602 is retained within the recess 410. The method includes applying a force to the piston 134 to move the center portion 128 to the resting position 122 (step 804). The center portion 128 is moved to the resting position passing the toggle position 144 thereby retaining the folding member 114 in a resting position keeping the force member 130 under load (step 806). The diaphragm member 132, with adhesives thereon, may be aligned and attached the retaining member 140. The retaining member 140 may releasably attach to the housing 102 to retain the folding member 114 and force member 130 therein.

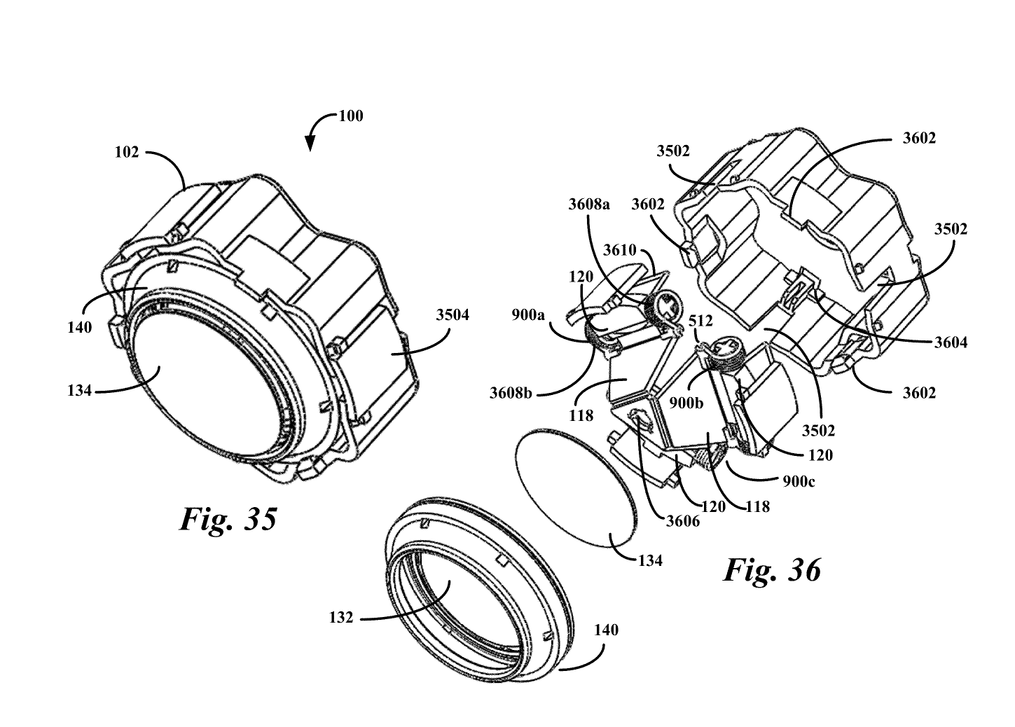

FIG. 35 schematically illustrates a bottom assembled view of an applicator according to a preferred embodiment of the invention. The applicator 100 includes the housing 102 with the retaining member 140 fixably attached thereto. The housing 102 includes a set of grooves 3502 (not shown—see FIG. 36) to align an alignment member 3504 of the folding member 114 to the housing 102. The piston 134 may be a rigid or flexible structure attached to the folding member 114 to impact, with the microneedles 108 of the diaphragm member 132, the stratum corneum 109. The piston 134 may have a diameter of 35.5 mm.

FIG. 36 schematically illustrates a bottom unassembled view of the applicator of FIG. 35. The applicator 100 is configured to house a folding member 114 that comprises a crank-slide assembly with three arms 116. The housing 102 is approximately 60 mm in wide and 28 mm in height. Each of the arms 116 is operatively attached to a torsional spring (900a, 900b, 900c). The housing 102 includes retaining hooks 3602 to connect to the retaining member 140 and a mounting member, as spring hook 3604, to retain each spring leg 3610 of the torsional springs 900.

The folding member 114 forms three symmetrically-shaped arms 116 attached to an equilateral triangular center portion 128. Each of arms 116 are configured as a four-bar linkage having (i) the first interlinking member 118 acting as a connecting link and (ii) the second interlinking member 120 acting as a crank link. The center portion 128 includes a mounting hole 3606 to retain the release member 142, which is shaped as a button and forms a unitary structure (not shown—see FIG. 38) with the piston 134. The diaphragm member 132 is attached to the retaining member 140 by attachment members 139 (not shown—see FIG. 38) and includes the microneedles 108 configured in the array 108a.

Each of the torsional springs (900a, 900b, 900c) forms two windings sections 3608a, 3608b connected to a U-shaped bar that forms the spring leg 3610, which attaches to the spring hook 3604 of the housing 102. The two ends of the torsional spring (900a, 900b, 900c) have an L-shaped hook that is retained in the hinge assembly 512 connecting the first and second interlinking members 118, 120.

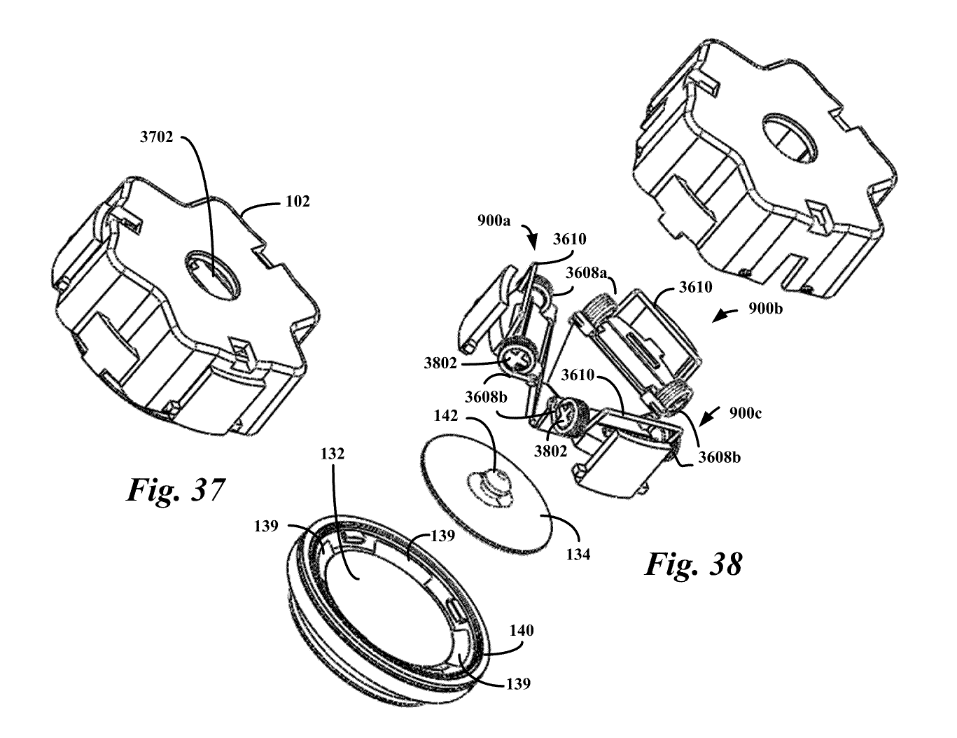

FIG. 37 schematically illustrates a top assembled view of the applicator of FIG. 35 in the de-energized state. The housing 102 is shaped as a lobed structure suitable to be held by the palm with four fingers, thereby leaving the index finger free to articulate with respect to the housing 102. An opening 3702 located in the center of the housing 102 allows for the release member 142 to protrude therethrough to be activated by the index finger. The opening 3702 is about 13.5 mm in width.

FIG. 38 shows other views of the torsional spring and schematically illustrates a top unassembled view of the applicator of FIG. 35. The second interlinking member 120 (e.g., crank link) have a pair of retaining pins 3802, which align with the longitudinal axis of the winding sections 3608a, 3608b of each of the torsional springs (900a, 900b, 900c). The torsion springs (900a, 900b, 900c), thus, form a three point contact with the spring hook 3604, the hinge assembly 512, and the retaining pin 3802 to rotate around the retaining pins 3802.

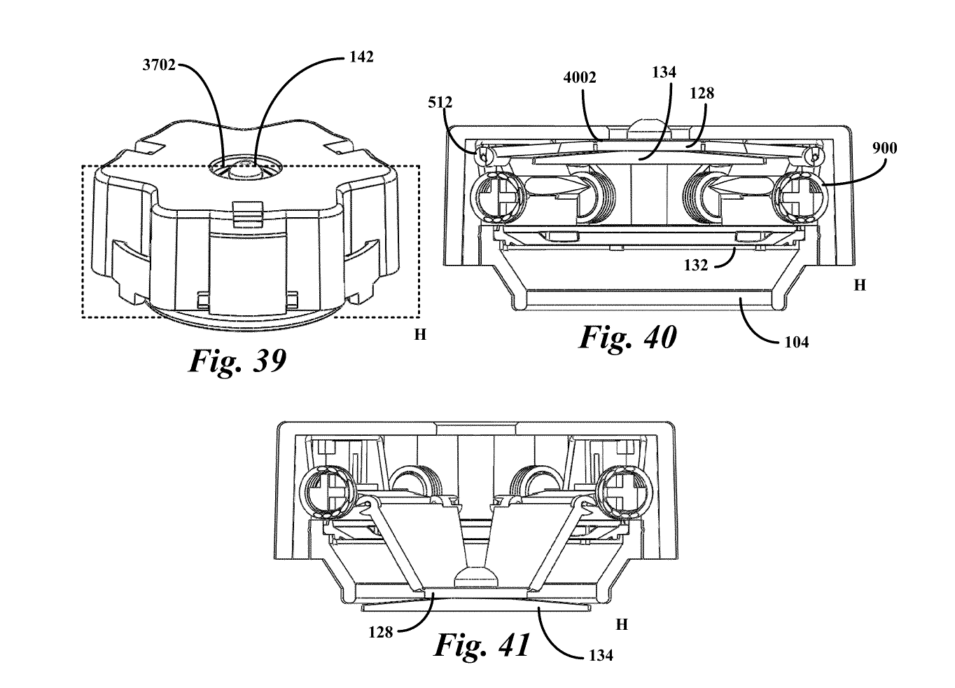

FIG. 39 schematically illustrates a top assembled view of the applicator of FIG. 35 in the energized state. The release member 142 extends from the opening 3702.

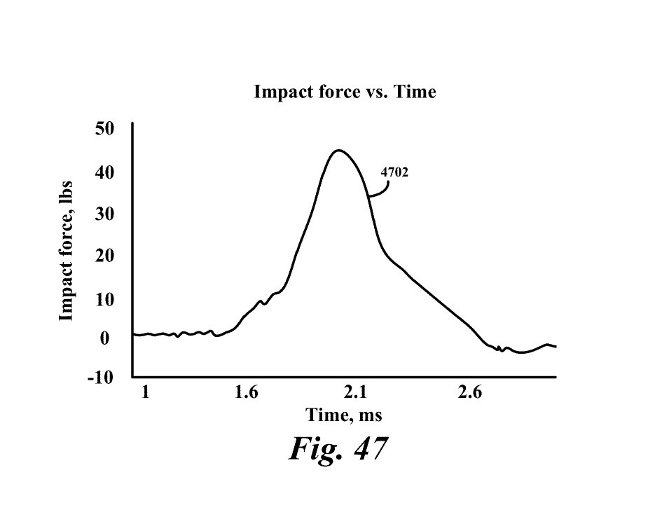

FIGS. 40-41 schematically illustrate cut-out views of the applicator of FIG. 35 in the energized and de-energized state. The cut-out view is defined by plane H shown in FIG. 39. In FIG. 40, the center portion 128 of the folding member 114 is in the resting position 122 and disposed against an inside surface 4002 of the housing 102 about 22.5 mm from the opening 104. The toggle position 144 is defined, in general, by a horizontal plane intersecting the hinge 512. Upon the release member 142 being displaced past the toggle position 144, the first interlinking member 118 (e.g., connecting link) allows the torsional spring 900 to rotate the second interlinking member 120 to propel the center portion 128 and piston 134 to the opening 104. Specifically, the center portion of the torsional spring 900 pushes against the housing 102 while the other portion pushes against the second interlinking member 120 (e.g., crank link). As the second interlinking member 120 rotates with the torsional spring 900, the center portion 128 is moved toward the opening 104. The piston 134 impacts the diaphragm member 132 to propel the frangible section with microneedles 108 thereon towards the stratum corneum 109. The center portion 128 stops at extended position 124 with the piston 134 passing the opening 104 when no impact surface is provided (see FIG. 41). When unloaded, the piston 134 extends less than 1 mm past the opening 104. An illustrative profile 4702 of the impact force is shown in FIG. 47. The various dimensions are merely exemplary and other dimensions and forces may be employed.

The embodiment shown in FIG. 1 (detailed drawings in FIGS. 35 to 41) was created and tested. The spring k values and velocity were measured as described above. The following results were obtained:

| | Spring Angular | | | | |
|---|---|---|---|---|---|
| Torsion | Displacements | | Measured | Calculated | |
| Spring | (radians) | | Spring | Energy (J) | Measured |
| Constant κ | Release | Extended | Constant | ½κθ_{Resting}² − | Velocity |
| (in-lb/rad) | State | State | (in-lb/rad) | ½κθ_{Extended}² | (m/s) |
| 0.99 | 2.37 | .22 | 0.80 | 0.26 | 8.38 |
| 1.34 | 2.37 | .22 | 1.24 | 0.35 | 10.53 |
| 1.64 | 2.37 | .22 | 1.75 | 0.43 | 11.93 |
| 1.98 | 2.37 | .22 | 1.79 | 0.52 | 12.28 |

This embodiment also incorporates a spring as shown in FIG. 4 (108) to normalize the release force. This release force is governed by the equation F=kx, where F is the release force, k is the spring constant, and x is the displacement of the spring from its neutral/zero position. The release force was designed to be 4 lbs., the release force was measured and found to be 4.3 lbs. on average.

The embodiments of the invention described above are intended to be merely exemplary; numerous variations and modifications will be apparent to those skilled in the art. All such variations and modifications are intended to be within the scope of the present invention as defined in any appended claims.

For example, transdermal agent delivery may also include delivery via passive diffusion as well as by external energy sources including electricity (e.g., iontophoresis) and ultrasound (e.g., phonophoresis). While drugs do diffuse across both the stratum corneum and the epidermis, the rate of diffusion through the stratum corneum is often the limiting step. Many compounds, in order to achieve a therapeutic dose, require higher delivery rates than can be achieved by simple passive transdermal diffusion.

The folding member 114 may have other numbers of arms 116, including four, five, six, seven, eight, etc.

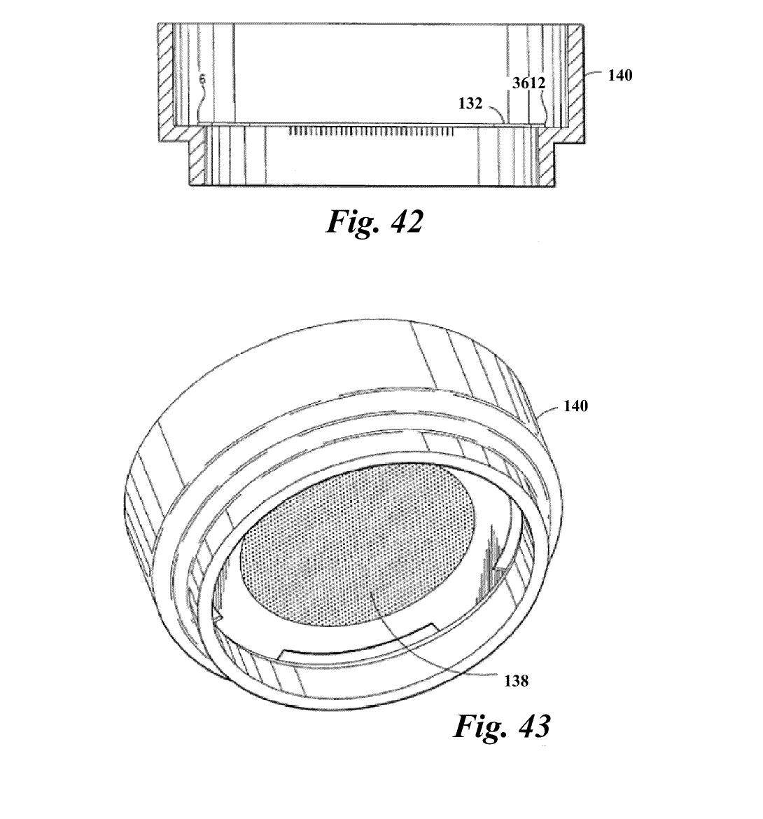

The retaining member 140 may be configured as a dock for use as a multiple use applicator. The dock has been described in U.S. Pat. No. 7,097,631, which is incorporated herein in its entirety, and shown in FIGS. 42-43.

What is claimed is:

1. An apparatus for impacting microneedles against the stratum corneum of a person, comprising:
a housing having an opening defining a first plane for impact against the stratum corneum;
a diaphragm member disposed within the housing at a second plane substantially parallel to the first plane, the diaphragm member having a plurality of microneedles;
a folding member fixably disposed within the housing, the folding member having a plurality of arms, each arm comprising a plurality of interlinking members to hinge-ably rotate between a resting position and an extended position; and
a force member operatively attached to the folding member to (i) store energy along a first direction when the plurality of interlinking members is in the resting position and (ii) move the plurality of interlinking members to the extended position when released from the resting position thereby moving the diaphragm member from the second plane to the first plane along a second direction transverse to the first direction,
wherein, when released from the resting position, the folding member translates energy stored within the force member to the second direction from the first direction.

2. The apparatus of claim 1, wherein the diaphragm member is fixably attached to the housing.

3. The apparatus of claim 1, wherein the diaphragm member is fixably attached to the folding member.

4. The apparatus of claim 1, wherein the plurality of interlinking members hinge-ably rotates via at least one of a hinge assembly and a flexible joint.

5. The apparatus of claim 1, wherein the folding member comprises at least three arms.

6. The apparatus of claim 5, wherein the folding member includes a body portion connecting to each of the plurality of arms, wherein the body portion is symmetrically shaped having a number of sides corresponding to a number of the plurality of arms.

7. The apparatus of claim 6, wherein each of the plurality of interlinking members includes a first member and a second member connected thereto, the first member being hinge-ably connected to the body portion, and the second member being hinge-ably attached to the housing.

8. The apparatus of claim 6, wherein each of the plurality of interlinking members includes a first member, a second member, and a third member, the first member being hinge-ably connected to the second member and the body portion, and the third member being hinge-ably connected to the second member and the housing.

9. The apparatus of claim 1, wherein the plurality of interlinking members includes a first member and a second member, the first member being generally horizontal while the second interlinking member being generally vertical when the plurality of interlinking members is at the resting position.

10. The apparatus of claim 1, wherein the force member operatively attaches to the folding member between a first member and a second member of the plurality of interlinking members, the first and second members being a part of an arm of the plurality of arms.

11. The apparatus of claim 1, wherein the force member operatively attaches to the folding member between a first member and a second member of the plurality of interlinking members, the first and second members being a part of different arms of the plurality of arms.

12. The apparatus of claim 11, wherein the first and second arms are neighboring.

13. The apparatus of claim 1, wherein the force member operatively attaches to the folding member and the housing.

14. The apparatus of claim 1 further comprising a second force member operatively attached to the folding member and the force member, the second force member being configured to store energy along the first direction when the plurality of interlinking members is in the resting position and to move, along with the force member, the plurality of interlinking members to the extended position when released from the resting position.

15. The apparatus of claim 6, wherein the force member operatively attaches to the housing and the body portion of the folding member.

16. The apparatus of claim 1, wherein the force member includes an energy storing member selected from at least one of a torsion spring, a coil spring, a planar leaf spring, a disc spring, a wave spring, and an elastic band.

17. The apparatus of claim 1, wherein the force member is in compression when the plurality of interlinking members is at the resting position.

18. The apparatus of claim 1, wherein the force member is in tension when the plurality of interlinking members is at the resting position.

19. The apparatus of claim 1 further comprising:
a release button disposed through the housing and in operative contact with the folding member, the release button having a retaining position corresponding to the resting position of the plurality of interlinking members.

20. The apparatus of claim 19, wherein the release button further comprises a releasing position, wherein movement from the resting position to the releasing position causes the force member to be further loaded prior to the force member being released.

21. The apparatus of claim 19, wherein the release button moves from the resting position to the releasing position at a force sufficient to normalize the stratum corneum for the microneedles delivery.

22. The apparatus of claim 1, wherein the release button comprises a locking portion.

23. The apparatus of claim 1, wherein the force member comprises a metallic material.

24. The apparatus of claim 1 further comprising:
a second force member operatively attached to the folding member to (i) store energy along a third direction, the first and second force members being attached to the same arm of the plurality of arms.

25. The apparatus of claim 1, wherein the folding member comprises a material including at least one of polyethylene and polypropylene.

26. A method of impacting microneedles against the stratum corneum of a person, comprising:
aligning, via a housing, a diaphragm member substantially parallel with the stratum corneum of a person, the diaphragm member having a plurality of microneedles;
releasing a folding member fixably disposed within the housing from a resting position to an extended position, wherein at the resting position, the folding member is operatively connected to a force member in a manner to place the force member under load in a first direction; and
propelling, via the force member, the folding member to an extended position thereby impacting a microneedle portion of the diaphragm member against the stratum corneum, the folding member being operatively connected to the force member to translate the release of the force member under load to a second direction traverse to the diaphragm member.

27. The method of claim 26, wherein the microneedles include a coating of a therapeutic agent or drug.

28. The method of claim 26, wherein upon the microneedle portion impacting the stratum corneum, microneedles disposed within the microneedle portion pierce the stratum corneum.

29. The method of claim 26 further comprising:
loading the force member at a second load greater than the load at the resting position before releasing the folding member.

30. The method of claim 26, wherein the folding member translates the release of the force member under load to impact the microneedles against the stratum corneum at an energy between 0.05 and 3 joules per cm² in less than ten milliseconds.

31. The method of claim 26, wherein the folding member translates the release of the force member under load to impact the microneedles against the stratum corneum at a velocity of at least three meters per second.

32. A method of manufacturing an apparatus for impacting microneedles against the stratum corneum of a person, the method comprising:
providing a folding member fixably disposed within a housing;
loading a force member operatively attached to the folding member to place the folding member in a resting position while the force member is under load in a first direction, the force member configured to move the folding member to an extended position along a second direction perpendicular to a plane of the stratum corneum; and
retaining the folding member in the resting position thereby keeping the force member under load.

33. The method of claim 32 further comprising:
aligning a diaphragm member in the housing along the plane of the stratum corneum, the diaphragm member having a plurality of microneedles.

34. The method of claim 33, wherein at least a portion of the plurality of microneedles includes a surface coated with a pharmaceutical agent.

35. The method of claim 34, wherein the pharmaceutical agent is adapted to be delivered transdermally by the plurality of microneedles.
